(19) United States
(12) Patent Application Publication
(10) Pub. No.: US 2012/0065567 A1
(43) Pub. Date: Mar. 15, 2012

(54) HEMODIALYSIS SYSTEM AND METHOD

(76) Inventor: Alfredo R. Zarate, Bethesda, MD (US)

(21) Appl. No.: 13/151,511

(22) Filed: Jun. 2, 2011

Related U.S. Application Data
(60) Provisional application No. 61/350,911, filed on Jun. 2, 2010.
(60) Provisional application No. 61/376,679, filed on Aug. 25, 2010.

(51) Int. Cl. A61M 1/14

(52) U.S. Cl. 604/5.04

(57) ABSTRACT

Hemodialysis systems and methods with streamlined blood flow paths are provided. Such streamlined blood flow paths facilitate flow without undue damage to circulating cells.

### CROSS-REFERENCE TO RELATED APPLICATIONS

The benefits of U.S. Provisional Application No. 61/350,911 filed Jun. 2, 2010 and entitled “Hemodialysis System and Method” and U.S. Provisional Application No. 61/376,679 filed Aug. 25, 2010 and entitled “Hemodialysis System and Method” are claimed under 35 U.S.C. §119(e), and the entire contents of these applications are expressly incorporated herein by reference thereto.

### FIELD OF THE INVENTION

Hemodialysis systems and methods are disclosed. More particularly, hemodialysis systems and methods with blood flow paths that avoid flow turbulence are disclosed, including the use of needles and/or catheters.

### BACKGROUND OF THE INVENTION

Currently there are about 400,000 end-stage renal disease (ESRD) patients on dialysis in the United States and about 1,600,000 world-wide, and their number is expected to grow exponentially. The medical complications (morbidity) experienced and the cost imposed by these patients is enormous. Most of the morbidity and the mortality of about 20% per year is derived from cardiovascular complications, and inflammation and oxidative stress plays a key role.

Hemodialysis is a medical treatment given to patients with kidney failure to remove substances from the blood that cannot be excreted by the failed kidneys. Hemodialysis can be given using needles or catheters. In hemodialysis with needles, blood is drawn from the patient by a needle called an “arterial needle”, circulated through a dialysis blood circuit that includes plastic tubes and an artificial kidney where the blood is cleaned, and then returned to the patient by another needle called the “venous needle.” A blood pump is attached to a segment of the plastic tubing of the dialysis circuit and is manually set to draw the desired amount of blood from the patient. The amount of blood drawn from the patient is called the dialysis blood flow rate and usually is 300-500 mL/min.

Alternatively, in hemodialysis with a catheter, single lumen and double lumen catheters are used. A single lumen catheter provides either of the suction and return paths, and typically is for short-term use for example for a few days. In particular, the single lumen catheter is a single, thin, plastic tube (e.g., formed of silicone) that can be used to suction blood from a patient (like an “arterial needle”) or return blood to the patient (like a “venous needle”). The double lumen catheter is a plastic tube with two thin tubes inside, one to suction blood (similar to the “arterial needle”) and the other to return cleaned blood (similar to the “venous needle”). In a double lumen catheter, blood enters the catheter (arterial side) via several holes extending through the tube. Blood entering through these holes travels within the arterial tube to an artificial kidney, where it is cleaned by the artificial kidney and then travels within the venous tube and exits at the tip of the catheter and at

These catheters can be inserted in a vein and kept inside the vein for days, weeks or months. Patients with acute kidney failure can receive four hours of dialysis daily for multiple days or a few weeks, until kidney function recovers, while patients with permanent kidney failure receive four hours of dialysis three times per week for an indefinite period of time or until they receive a kidney transplant or die.

Such catheters are available in different sizes (outer diameters or “OD”), and one of the largest is 16 Fr size (1 Fr=0.33 mm OD, 15 Fr=5.0 mm OD, 16 Fr=5.3 mm OD). Catheters for insertion in a femoral vein typically are about 12 inches long (with 6 inches provided for insertion into tissues and with the tip usually reaching the Iliac veins). Catheters to reach the cava veins typically are up to 36 inches long and are provided to extend close to the heart and even into the right atrium. Finally, a “Tesio” venous catheter is formed of two separate 10 Fr catheters (each 3.3 mm OD; about 2.0 mm inner diameter).

Blood circulates during dialysis through some segments of the currently-used dialysis blood circuit at extremely high velocity and turbulence, and this causes the activation of mononuclear cells, along with the generation and release of proinflammatory cytokines and oxidants that have been identified as causing high morbidity and mortality of dialysis patients. Thus, there exists a need for a dialysis blood circuit that permits lower velocity and turbulence such that lower morbidity and mortality rates can be achieved.

The current dialysis blood circuit is formed of 16-18 feet of plastic tubing connected to an artificial kidney and to the patient. With reference to FIGS. 1-2, the circuit includes an arterial fistula needle 1 that has two components, a metal cannula 1a and a plastic segment 1b called an arterial needle plastic segment or fistula needle tubing that is about 12 inches long. The circuit also includes an arterial blood line 4 (with a port 4a) that is about 6-8 feet long, an artificial kidney 10 with an arterial side portion 10a and a venous side portion 10b, a venous blood line 11a, 11b (with a port 11c) that is about 6-8 feet long and has an air trap chamber 16, and a venous fistula needle 19 that is similar to the construction of the arterial fistula needle and is formed of a metal cannula 19a and plastic segment 19b.

The arterial fistula needle 1 suctions blood from the patient, while the arterial blood line 4 takes this blood to the artificial kidney 10. The venous blood line 11a, 11b takes the cleaned blood from artificial kidney 10 to the venous fistula needle 19 which in turn returns the cleaned blood to the patient. Artificial kidney 10 is formed for example of a few thousand micro-tubes of very small diameter and about 12 inches in length, the micro-tubes being bundled together and positioned in parallel. An exemplary artificial kidney has a cylindrical shape. Blood enters artificial kidney 10 through an “arterial port” 8 and then enters each one of the thousand micro-tubes where it is cleaned and exits artificial kidney 10 through a venous port 12 located at the other end thereof.

As will be further described below, numerous connection regions in the circuit present diameter changes and/or sharp corners or bends including: connection region 3 between metal cannula 1a and plastic segment 1b, connection region 7 between plastic segment 1b and fistula needle tubing 5 (which typically may include a free end segment or connector 5, such connectors for example usually having luer locks), connection region 6 between arterial blood line 4 and arterial port 8, connection region 14 between venous port 12 and venous blood line 11a (which may include a free end segment or connector 11d), connection region 15 between venous blood line 11b and plastic segment 19b (similar to connection region 7), and connection region 20 between plastic segment 19b (also called fistula needle tubing) and metal cannula 19a (similar to connection region 3). Moreover, regions 16a, 16b of air trap chamber 16 present diameter changes and/or sharp corners or bends.

In the prior art, connection regions 7, 11d, for example, are Luer lock types of connectors that couple components to one another and resist separation. Such a prior art connector typically has an internal diameter that is smaller than the internal diameters of the components being coupled. Thus, neither a smooth transition between the components nor a uniform internal diameter from one component to the next are provided. Such connectors can substantially contribute to increases in blood velocity and turbulence in the hemodialysis circuit.

Unlike in normal anatomical vessels, blood circulates through the current designs of the dialysis circuit at extremely high velocity and turbulence. This is because of a variety of factors, including: (1) the blood flow rate is high, (2) several segments of the circuit have a very small diameter (which increases the velocity), (3) the diameter changes from large to small which increases the velocity and causes turbulence, or from small to large which decreases the velocity and causes turbulence, (4) there are sharp corners or bends that cause flow separation or localized turbulence, and (5) there are irregularities in the internal wall of the circuit that protrude into the lumen of the circuit and cause turbulence and damage to cells that impact on the irregularities. Moreover, the excessive length of the blood circuit increases the surface where circulating cells contact and are activated. In addition, unlike the endothelium, the circulating cells experience friction against the wall of the circuit. The velocity of blood inside many segments of the current blood circuit is much higher than the velocity in normal arteries and veins.

Table I shows a comparison of the velocity of the post venous needle flow (PVNF) with the velocity of the flow in various vessels in the body (data from various sources including literature data, calculated data, and measured data from work at the Georgia Institute of Technology). A comparison of needles and catheters is included. The calculated mean velocity is determined from the flow rate/cross-sectional area (or πr²). However, because the flow of blood during dialysis is peristaltic, the mean flows and velocities are actually higher than shown in Table I.

TABLE I

| Location | Flow Rate | | Velocity | |
|---|---|---|---|---|
| Saphenous vein | 10-70 | mL/min | 0.07-0.3 | m/s |
| Dialysis graft, off dialysis | 1.0 | L/m | 0.59 | m/s |
| Blood in a dialysis vascular access after | 1-1.5 | L/min | 3-7 | m/sec |
| leaving the venous needle (access flow | | | | |
| 1 L/m, needle flow 300-600 mL/m, | | | | |
| 15 G needle measured with laser | | | | |
| Doppler velocimetry) | | | | |
| Femoral artery | 350 | mL/min | 0.05-0.8 | m/s |
| Aortic root | 5 | L/min | 1.0-1.5 | m/s |
| Inside current dialysis blood lines | 600 | mL/min | 0.8 | m/s |
| (4 mm internal diameter) | | | | |
| Inside current connector between the | 600 | mL/min | 3.1 | m/s |
| blood lines and the fistula needle | | | | |
| tubing (2 mm internal diameter) | | | | |
| Inside the metal cannula, 15 G | 600 | mL/min | 4.2 | m/s |
| Inside the metal cannula, 16 G | 600 | mL/min | 5.0 | m/s |
| Inside venous tube of a double lumen | 400 | mL/min | 5.6 | m/s |
| dialysis catheter, 15 Fr (4.5 mm overall | | | | |
| catheter outer diameter, 2.25 mm outer | | | | |
| diameter per lumen, about 0.5 mm wall | | | | |
| thickness per lumen, about 1.25 mm | | | | |
| internal diameter per lumen (calculated | | | | |
| velocity) | | | | |
| Inside venous tube of double lumen | 400 | mL/min | 3.7 | m/s |
| dialysis catheter, 16 Fr (4.8 mm overall | | | | |
| catheter outer diameter, 2.4 mm outer | | | | |
| diameter per lumen, about 0.5 mm wall | | | | |
| thickness per lumen, about 1.4 mm | | | | |
| internal diameter per lumen (calculated | | | | |
| velocity) | | | | |

The mean value of the velocity at the aortic root, as determined by Doppler, was 1.01 m/s. See Robson, D. J. et al., Intensive Care Medicine 11 (1995), 90-91. At a flow rate of 600 mL/min, the velocity inside a 15G cannula is 80 times higher than the velocity in a femoral artery and the velocity inside a 16G cannula is 100 times higher.

The circulation of blood through dialysis catheters may be more harmful than the circulation through dialysis needles for several reasons. First, blood circulates through the venous tube of the dialysis catheter at higher velocity and higher shear stress because the internal diameter of dialysis catheters is smaller than the internal diameter of the dialysis needle. Moreover, blood circulates through a longer segment of catheter than venous needle: 25 cm to 75 cm in length of catheter, versus 2.5 cm which is the length of a needle. Second, blood exits the venous end of a dialysis catheter at extremely high velocity and turbulence. For example, a 16 Fr double lumen catheter, the largest size double lumen catheter available (with an outer diameter of about 4.8 mm), has two thin catheters inside (arterial and venous). The outer diameter of the venous and arterial catheters is 2.4 mm, and because the wall thickness of each catheter is about 0.5 mm, the internal diameter of the venous catheter is about 1.4 mm which is smaller than the internal diameter of a 15G fistula dialysis needle (about 1.75 mm). Blood exiting this opening at a flow rate of 400 mL/min would exit at a calculated velocity of 3.7 m/s. The distal end of some existing tapered catheters has an internal diameter of about 1.0 mm, and with a flow rate of 400 mL/min the calculated velocity of the blood exiting the catheter would be 7.1 m/s. Blood exits a venous end of a 15 Fr catheter (1.25 mm internal diameter) at a velocity of 5.6 L/min at a flow rate of 400 mL/min.

For comparison, as shown in Table I, a velocity of 5.6 m/s is about 1.3 times higher than the calculated mean velocity inside a 15G fistula needle (4.2 m/s), 5 times higher than the velocity in the aorta (the highest velocity in the body) and about 16 times higher (systole) and 71 times higher (diastole) than the velocity inside a saphenous vein. In turbulent flow, like the one present after blood exits the venous catheter, any increase in the velocity of the flow causes an exponential increase in the turbulence because turbulence (measured as turbulent intensity) is proportional to the square of the fluctuating velocity.

The high velocity and turbulence of the blood inside the current dialysis circuit cause activation or destruction of circulating blood cells. In activated white cells, genes are up-regulated to increase the generation and release of pro-inflammatory cytokines and oxidants, causing a state of inflammation and oxidative stress. Inflammation and oxidative stress are believed to cause hypertension, anemia, heart failure, coronary artery disease, hyperphosphatemia, and malnutrition which cause or contribute to cause the 20% per year mortality of patients with permanent kidney failure.

Because cells make up about 40% of the blood volume, billions of cells circulate through the blood circuit and can be activated or destroyed during a single dialysis. If the blood flow rate is 450 mL/min, the volume of blood that is drawn from and returned to the patient is about 108 L of blood that circulates through the circuit during a single 4 hour dialysis. In a patient with a circulating blood volume of 4.0 L, all the blood and the blood cells in the body would circulate through the dialysis circuit every 9 minutes for 26 times, that is, about 650 million to 1.3 billion leucocytes, 432 billion erythrocytes and 16 billion platelets.

Because hemodialysis is given every other day to patients with permanent kidney failure, and the inflammatory and oxidative stress that is caused by dialysis lasts for several hours, the repetitive damage to cells during each dialysis could lead to a state of chronic (permanent) inflammation and oxidative stress and high morbidity and mortality.

Therefore, dialysis catheters can cause more activation of circulating cells due to higher velocity and shear stress inside the catheter as well as the higher velocity and turbulence after exiting the venous catheter which increases generation and release of proinflammatory cytokines and oxidative stress. In addition, excessive activation of platelets and repetitive damage to the endothelium of the vein can cause thrombus formation or stenosis of the vein.

The current design of the dialysis blood circuit does not fulfill the needs of hemodialysis as delivered to patients with permanent kidney failure at present. Hemodialysis for those patients requires the circulation of a large volume of blood (high flow rates) three times a week for four hours per session. There is a need to minimize the damage to circulating mononuclears. But, in the current dialysis circuit, blood circulates at high velocity and turbulence, is exposed to walls of the circuit that are made of materials not resembling the endothelium of vessels, and the circuit is about 18 feet long, all of which are factors that can contribute to damage to circulating mononuclears.

The circulation of fluids inside tubes of different diameter, length, roughness, bends, and flow rates is well described in the engineering literature. See, e.g., Munson, B. R. et al., Eds., Fundamentals of Fluid Mechanics, New York, John Wiley & Sons. (1990), pp 484-547; Schlichting, H., Ed., Boundary Layer Theory, 4th Ed., New York, McGraw-Hill (1960), pp. 502-506. This knowledge can be applied to understand the circulation of blood in the dialysis circuit, keeping in mind that blood carries cells and in the current circuit, blood circulates at high velocity and turbulence, while the cells are exposed to foreign surfaces that can harm the circulating cells.

We next turn to the fluid dynamics (hemodynamics) inside the blood dialysis circuit. The blood flow is laminar (blood flows on parallel streamlines) in segments of the blood circuit where the velocity is not very high, the diameter is uniform and large, the surface is smooth, and there are no curves, sharp angles, or bends. Laminar flow (and laminar shear stress) causes less activation or damage to circulating cells because the velocity of the flow is not very high and the cells travel in the blood in straight streamlines. In laminar flow, the shear stress (friction) increases proportionally to any increase of the mean velocity of the flow.

The blood flow is turbulent (chaotic motion of the blood, vortices and eddies are formed that move at random tri-dimensionally) in segments of the blood circuit where the velocity is very high, there are irregularities protruding inside the lumen of the circuit, the diameter of the circuit changes from large to small and vice versa, there are bends or sharp turns, and/or the wall of the circuit is rough. Turbulent flow (and turbulent shear stress) is more harmful than laminar flow because the velocity of the blood is higher (there is more friction) and because of the chaotic motion of blood. The cells inside the vortices and eddies impact on each other or on the walls of the circuit, causing greater activation or damage to the cells. In turbulent flow, cells in the blood behave like “missiles” colliding at high velocity against the circuit wall or against other cells. Also, in turbulent flow, the shear stress increases exponentially with any increase in the velocity of the flow because it is proportional to the square of the fluctuating velocity.

Changes in diameter of tubes, sharp corners, and bends cause changes in the velocity and flow pattern of blood in the dialysis circuit. Any increase in the diameter of the circuit (deceleration of the flow), decreases in the diameter of the circuit (acceleration of the flow), bends, and sharp corners cause flow separation from the wall and can be damaging to circulating cells. Such flow separation is shown, for example, in FIGS. 7A and 7B herein (from Munson, B. R., supra, p. 512). Flow separation is an area of localized turbulence close to the wall unlike well developed turbulent flow in which turbulence is present in the entire cross-section of the circuit.

As shown in FIG. 3 (related to single-phase flows; from Cheremisinoff, N. P. et al., Handbook of Fluids in Motion, Ann Arbor Science (1983), pp. 29-67) and FIG. 4 (from Munson, B. R., supra), when blood flows from a region of a first diameter to a region of a second diameter larger than the first diameter, the velocity decreases, the fluid separates from the wall, and it expands to occupy all the cross-section of the larger conduit. The region between the expanding jet and the wall is filled with fluid in vortex motion, and an area of recirculation/flow separation or localized turbulence is formed because fluids cannot take a sharp angle. The vortices of flow separation cause chaotic movement of circulating cells and impact of cells on the wall. Also, there is turbulence because the plasma would slow down before the cells in circulation and the cells up front would decelerate before the cells behind so that these cells would impact on each other.

As shown in FIG. 5 (from Cheremisinoff, N. P., supra) and FIG. 6 (from Munson, B. R., supra), blood instead can flow from a region of a first diameter to a region of a second diameter smaller than the first diameter. Before blood leaves a tube of larger diameter and enters a tube of smaller diameter, it forms an area of flow separation/turbulence inside the larger diameter region where cells impact on the wall of the larger diameter and the small diameter tubes.

When the fluid enters a smaller diameter tube, a vena contracta is formed because fluids cannot flow at sharp right angles. In the vena contracta, the flow separates from the wall and the cells behave like in turbulent flow (chaotic random motion). In the vena contracta, the velocity of the fluid at the center of the tube is higher than the downstream flow even if the diameter of the pipe is the same because the vena contracta is of annular shape and decreases the diameter of the region where the main flow circulates.

As shown in FIGS. 3-6, sharp corners and bends can be encountered. Before blood enters a smaller diameter tube or a larger diameter tube, if the connection or transition has sharp corners, there is a region of recirculation where cells move at random like in fully developed turbulence because fluids cannot take sharp corners with perfectly laminar flow. Likewise, bends in the circuit can cause flow separation. See, e.g., Munson, B. R., supra, pp. 512.

It will be appreciated that each segment of the blood circuit can have particular hemodynamics characteristics. With respect to an arterial fistula needle, the metal cannula typically has an internal diameter between needle sizes 14G to 17G, e.g., 1.75 mm (needle size 15G) or 1.6 mm (needle size 16G). The fistula needle tubing has an internal diameter of approximately 3 mm. The metal cannula is inserted into the lumen of the tubing where it fits tightly. Because the cannula has a smaller diameter than the plastic tubing, the rim of the cannula protrudes in the lumen of the tubing. Blood circulates at the highest velocity inside the cannulas and causes the highest friction. The calculated mean velocity of the blood inside a 15G metal cannula is 4.1 m/s at a blood flow rate of 600 mL/min and the calculated mean velocity of the blood inside a 16G cannula is about 5 m/s (see, e.g., Table I). It should be noted that the calculated mean velocity is the flow rate/cross-section or the flow rate/πr². Blood circulates at a lower velocity of 1.4 m/s inside the fistula needle tubing of 3 mm internal diameter if the flow rate is 600 mL/min. When blood leaves a tube of smaller diameter (cannula) and enters a tube of larger diameter (fistula needle tubing), an area of recirculation/flow separation and turbulence is formed, as shown for example in FIGS. 3-4, because fluids cannot take a sharp angle, and cells collide with each other.

With respect to an arterial blood line, for example as shown in FIG. 1, the arterial blood line has a larger internal diameter (4 mm) than the fistula needle tubing (3 mm). One end of arterial blood line 4 connects with fistula needle tubing 1b (3 mm internal diameter) and the other end connects with the arterial port 8 of artificial kidney 10 (2 mm internal diameter). Blood circulates inside blood line 4 (internal diameter of 4 mm) at a calculated mean velocity of 0.8 m/s at a flow rate of 600 mL/min. The fistula needle tubing 1b (3 mm internal diameter) is connected with arterial blood line 4 (4 mm internal diameter) by a connector 5 (a 5-10 mm long segment of hard plastic tubing of 2 mm internal diameter) that is interposed between fistula needle tubing 1b and the blood line tubing 4. The rim of the connector protrudes inside the lumen of fistula needle tubing 1b and inside the lumen of the blood line tubing 4. When blood leaves fistula needle tubing 1b (3 mm internal diameter) and enters connector 5 (2 mm internal diameter), an area of flow separation/turbulence is formed inside the connector which is called vena contracta, as shown in FIGS. 5-7.

The blood circulates inside connector 5 at a velocity of 3.1 m/s at a blood flow rate of 600 mL/min. Cells are damaged by the high friction caused by the high velocity and turbulence. Also, cells coming from fistula needle tubing 1b are damaged when they impact on the rim of the connector 5. Impact with the rim can cause damage to many circulating cells: in one existing product, the external diameter of the connector is 3 mm and the internal diameter is 2 mm. That is, about 33% of the blood covering the cross-section of fistula needle tubing could impact on the rim of the connector.

When blood leaves connector 5 and enters the larger diameter of blood line 4, an area of flow separation and turbulence is formed.

The end of blood line 4 that connects with arterial port 8 of artificial kidney 10 has an internal diameter of 6 mm and the blood circulates at a calculated mean velocity of around 0.35 m/s at a blood flow rate 600 mL/min. The arterial port of one commercially available artificial kidney (Gambro) has a larger internal diameter of 8 mm so the velocity is slower inside this segment but flow separation/turbulence occurs because of the sudden increase in the diameter similarly to what happens in the cannula/fistula needle tubing connection. The arterial port of another commercially available artificial kidney (Fresenius) has a connector with an internal diameter of 4 mm and blood circulate through this connector at a velocity of 0.8 m/s at a blood flow rate of 600 mL/min. In this case, there is a gap between the blood line and the connector of larger internal diameter where turbulence also occurs.

Some arterial blood lines have “ports,” such as port 4a as shown in FIG. 1, which are used to measure pressures, to inject medications, or to extract blood.

Some arterial blood lines also have air trap chambers interposed in the circuits which are similar to the air trap chamber 16 present in the venous circuit such as shown in FIG. 2. These air traps have a larger diameter and rectangular or cylindrical shape. In some existing products, the connection of the blood lines with the air trap has a smaller diameter than the blood lines where the velocity (and friction) of the blood increases.

In one example, a 12 inch long segment of arterial blood line has an 8 mm internal diameter and is the segment that is compressed by the blood pump to move the blood forward. Blood enters this 8 mm diameter segment from the 4 mm diameter blood line, and the blood flow expands which causes flow separation and slower velocity. When the blood leaves this segment, after the rollers have compressed the tube, the blood has higher velocity and turbulence, and then enters the 4 mm diameter blood line where the velocity and turbulence increases.

As shown in FIGS. 1-2, blood enters artificial kidney 10 through an arterial port 8 located at one end of the cylindrically shaped artificial kidney and exits artificial kidney 10 through a venous port 12 located at the other end of artificial kidney 10. The velocity of the blood inside the microtubules of the artificial kidney is very low because a small volume of blood circulates through each microtubule, but friction of cells with the wall of the micro-tubes may activate circulating cells.

The plastic tubing of venous blood line 11a, 11b has similar length and internal diameter (4 mm) to arterial blood line 4, but the blood flows in opposite direction, from the artificial kidney 10 to the fistula needle 1. One end of the venous blood line 11a connects to the venous port 12 of artificial kidney 10 and the other end connects with the fistula needle tubing 19b of the venous fistula needle 19. In Gambro blood lines, since blood flows from a larger diameter venous port of the artificial kidney (8 mm) to a smaller diameter blood line (6 mm), flow separation/turbulence develops and it is possible that a vena contracta is formed. Also, blood cells impact on the rim of the blood line. Because the external diameter of the blood line is 8 mm and the internal diameter is 6 mm, while the internal diameter of the arterial port is 8 mm, the 1 mm wall of the blood line protrudes into the lumen. That is, about 25% of the blood cells flowing through the cross-section could impact on the rim and get damaged. The venous port of the Fresenius artificial kidney has a connector with an internal diameter of 4 mm and blood circulates through this connector at a velocity of 0.8 m/s at a blood flow rate of 600 mL/min. In this case, there is a gap between the blood line and the connector of larger internal diameter where turbulence also occurs.

As shown for example in FIG. 2, a connector 11d can be used between venous blood line 11b and fistula needle tubing 19b. Similar to the arterial blood line, the tubing of venous blood line 11b, 11d (internal diameter of 4 mm) connects with fistula needle tubing 19b (3 mm internal diameter) by a connector 11d of 2 mm internal diameter interposed between fistula needle tubing 19b and the blood line 11b, and the blood has the same velocity as inside connector 5 in arterial blood line. Because blood flows from the larger diameter blood line to a smaller diameter connector, flow separation/turbulence occurs and a vena contracta may form inside the connector.

The venous blood line 11a, 11b also may have an air trap chamber 16 interposed in the circuit. In an existing product, the openings to the air trap may have a smaller diameter than venous blood line 11a, 11b, and this increases the velocity of the blood in those regions. Some venous blood lines also have “ports” such as port 11c along the lines to measure pressures, to inject medications, or to extract blood.

With respect to a venous fistula needle 19, this has similar length, internal diameter, and velocities of the blood to the arterial fistula needle, but the blood flows in opposite direction, from the fistula needle tubing 19b of larger diameter to the metal cannula 19a of smaller diameter. This also can form a vena contracta. In addition, circulating cells can be damaged by impacting on the rim of the metal cannula. Because the thickness of the wall of metal cannula 19a is 0.1-0.2 mm, the rim takes about 11-22% of the entire cross-section of the connector. This means that 11-22% of the blood going through the cross-section of fistula needle tubing 19b could impact on the rim of metal cannula 19a.

Turning to FIG. 8, a rotating blood pump 30 also can be a major cause of high pressure, velocity and turbulence with associated activation or destruction of circulating cells. The commercially available blood pumps have 2-3 rollers 32 mounted in a rotating head that move clockwise in circular motion. Rollers 32 move the blood forward by progressively compressing tube 34 in a forward, clockwise direction. The velocity, turbulence and pressure of the blood progressively increases as tube 34 is progressively compressed because the internal diameter is progressively decreased as shown for example in region 36 (compression phase) and the velocity, turbulence and pressure of the blood progressively decreases because the internal diameter of tube 34 is progressively increased during the decompression phase. FIG. 8B shows the variation in blood velocity as a function of compression of the tubing in which the blood flows by the blood pump rollers. The alternating cycles of compression and decompression give a pulsatile or peristaltic flow (unlike steady flow in which the flow rate and velocity of the fluid does not change). For example, the segment of tube that is exposed to the rollers in a commercially available blood circuit has an internal diameter of 8 mm. Each time a roller 32 “compresses” the plastic tubing, the internal diameter of tube 34 is progressively decreased from 8 mm to zero for fractions of a second and when a roller 32 “decompress” the tube 34, the internal diameter increases from zero to 8 mm, the original internal diameter, in fractions of a second. Each time a roller 32 compresses the tubing, blood is propelled faster, and when roller 32 stops compressing tube 34 the velocity and blood flow rate decrease.

The blood pumps such as pump 30 are volumetric pumps that increases the blood flow rate by increasing the rate of rotation of the rollers/minute (rpm); higher rpm increases the frequency of compression/decompression of the tube, the frequency of increase and decrease in the velocity/turbulence/pressure, and the damage to circulating cells. For example, at a blood flow rate of 400 mL/min, each roller of an existing dialysis blood pump with two rollers compresses the tubing about 80 times per minute or about 40,000 times during a four hour dialysis. That is, the blood velocity, turbulence and pressure increase and decrease about 40,000 times during one dialysis and each cycle of compression and decompression causes cell damage and activation. When the internal diameter of the tube is 8 mm, the velocity of the fluid is 0.15 m/s, and when the tube internal diameter is compressed to 1 mm, the velocity is 7.5 m/s as shown in FIG. 8B.

Blood moves along the dialysis circuit because of a gradient pressure. During each cycle of compression/decompression of the tubing, certain changes in the hemodynamics occur. Each time the roller progressively compresses the tube, the diameter of the tubing decreases, the velocity of the blood inside the tubing increases, and turbulence is caused by the change in internal diameter and change in velocity. The high velocity of the blood at the end of each compression decreases because of the resistance to the flow caused by the blood downstream of the roller, the geometry of the dialysis circuit, and the semi-rigidity of the tube walls. When the velocity decreases, the pressure increases inside the tubing as per the Bernoulli's equation. This increase in pressure is what propels the blood forward. Turbulent flow causes higher resistance than laminar flow. If the resistance is measured as a pressure drop, the resistance (or pressure drop) in laminar flow is proportional to the mean velocity of the flow (P1−P2=mean velocity), whereas in turbulent flow the resistance is proportional to the mean velocity^{1.75} (P1−P2=mean velocity^{1.75}). See, e.g., Schlichting, H., supra, pp. 502-506. Therefore, much kinetic energy is wasted in turbulent flow.

Each time the roller progressively decreases the compression (decompression phase), the diameter of the tube increases, the velocity of the blood decreases, the pressure in the tubing increases, but turbulence persists at lower velocity because of the changes in diameter and velocities of the flow.

In hemodialysis with a catheter, the same blood circuit is used except that the arterial blood line and the venous blood line are connected to an arterial port and venous port of the catheter using connectors with the same geometry as the connectors of the blood lines described above.

The basic mechanism for activation or destruction of cells during dialysis should be understood. Cells can be activated during dialysis by several mechanisms including the formation of platelet-leucocyte aggregates, contact with dialysis membranes via the complement and the coagulation pathways, contact with bacterial endotoxins present in dialysate, contact with circulating cytokines, antioxidants or angiotensin and shear stress. See, e.g., Raj, D. S. C. et al., “Association of soluble endotoxin receptor CD14 and mortality in maintenance hemodialysis patients,” Am. J. Kidney Dis.; Sitter, T. et al., “Diaysate related cytokine induction and response to recombinant human erythropoietin in HD patients,” Nephrol. Dial. Transpl., Vol. 15, pp. 1207-1211 (2000); Port, F. K. et al., “The role of dialysate in the stimulation of interleukin-1 production during clinical haemodialysis,” Am. J. Kidney Dis., Vol. 10, pp. 118-122 (1987); Summers, D. S., “Essential hemodynamic principles,” in Rutherford, R. B., Vascular Surgery, WV Saunders Co., 1994, pp 18-11 and 411; Raj, D. S. C. et al., “Interleukin-6 modulates hepatic and muscle protein synthesis during hemodialysis,” Kidney Intern., Vol. 73, pp. 1054-1061 (2008); Raj, D. S. C. et al., “Skeletal muscle, cytokines and oxidative stress in End-Stage Renal Disease,” Kidney Int., Vol. 68, pp. 2338-2334 (2005); Raj, D. S. C. et al., “Hemodialysis induces mitochondrial dysfunction and apoptosis,” Eur. J. Clin. Invest., Vol. 37, pp. 971-977 (2007); Raj, D. S. C. et al., “Markers of inflammation, proteolysis and apoptosis in ESRD,” Am. J. Kidney Dis., Vol. 42, pp. 1212-1220 (2003); Raj, D. S. C. et al., “Association of soluble endotoxin receptor CD14 and Mortality in Maintenance Hemodialysis Patients,” Am. J. Kidney Dis.

In shear stress, the cells are activated by friction against the wall of the dialysis circuit (i.e. the inside of needles), against the wall of the dialysis vascular access post venous needle or with other cells (inside the dialysis circuit or inside the vascular access). High shear stress is probably the most important cause of cell activation or destruction during dialysis with high blood flow rate. High shear stress is the cause of the minimal hemolysis associated with standard dialysis and the cause of serious hemolysis reported in the literature. See Ming-Chien, Yan, “In vitro characterization of the occurrence of hemolysis during extracorporeal blood circulation,” ASAIO Journal, Vol. 46(3): 293-297, May-June 2000; Sweet, S. J. et al., “Hemolytic reactions mechanically induced by kinked HD lines,” Am H J Dus 27(2): 262-66 (February 1996); Dhaene, M. et al., “Red blood cell destruction in single-needle dialysis,” Clin Nephrol 31(6): 327-31 (June 1989); Kameneva, M. V. et al., “In vitro evaluation of hemolysis and sublethal blood trauma,” ASAIO J. 48(1): 34-8 (January-February 2002); Duffy, R. et al., “Multistate outbreak of hemolysis in hemodialysis patients traced to faulty blood tubing sets,” Kid Int Vol 57(4), pp. 1668 (April 2000); Gault, M. H. et al., “Hemolytic reactions mechanically induced by kinked hemodialysis lines,” Am. J. Kidney Dis. 27(2), pp. 262-266 (1996). The earliest changes that shear stress causes on cells occur in the up-regulation of genes which occurs about two hours after the mechanical force is applied. The gene expression has been studied in various cell and demonstrated in dialysis by Raj, D. S. et al., “Hemodialysis induced mitochondrial dysfunction and apoptosis,” Eur. J. Clin. Invest. 37, pp. 971-977 (2007).

Shear stress and turbulent flow are a cause of activation of cells during dialysis. The flow in normal blood vessels is laminar and laminar flow does not damage circulating cells or the endothelium because the velocity of the flow is low and the cells travel in the blood in straight streamlines. During dialysis, turbulent flow of high velocity is present inside the vascular access in the area past the venous needle and this turbulent flow (and turbulent shear stress) is harmful to the endothelial and circulating blood cells because the cells experience friction with each other and impact on each other or on the wall of the vascular access causing more significant activation or destruction of cells.

High shear stress causes cell activation and genes expression. See Ling, X. et al., “Dynamic investigation of leukocyte-endothelial cell adhesion under fluid shear stress in vitro,” Acta Biochim. et Biophys. Sinica. 35(6): 567-72 (June 2003); Huddleson, J. P. et al., “Fluid shear stress induces endothelial KLF2 gene expression,” Biological chemistry. 385(8): 723-9 (August 2004); Fukuda, S. et al., “Mechanisms of regulation of fluid shear stress response in circulating leukocytes,” Circulation research 86(1): E13-8 (Jan. 7, 2000); Sheikh, S. et al., “Exposure to fluid shear stress modulates the ability of endothelial cells to recruit neutrophils in response to TNF-a,” Blood 102(8): 2828-34 (Oct. 15, 2003); Pomianek, M. J. et al., “Synthesis of TNF-a is enhanced by exposure of blood to shear stress,” ASAIO J. 42(1): 52-9 (January-February 1996); Zhang, Jian-ning et al., “Duration of exposure to high fluid shear stress is critical in shear-induced platelet activation-aggregation,” Thromb Haemost 90: 672-8 (2003).

Turbulence is measured as turbulence intensity=[(u′)²]^{1/2} or root-mean-square of the fluctuating velocity u′. In turbulence flow, an increase of the velocity of the flow causes a proportional increase of the fluctuating velocity u′ and an exponential increase of the turbulence intensity.

Turbulent flow does not occur in normal vessels because it is an inefficient way to transfer fluids (⅓ higher consumption of energy than laminar flow), it would require much higher arterial pressure to propel blood in normal vessels, and turbulent flow, unlike laminar flow, is harmful to cells. Shear stress in laminar flow is proportional to the mean velocity of the flow, whereas shear stress in turbulent flow (i.e. post venous needle flow) is proportional to the square of the velocity. Shear stress in turbulent flow=⅛×lambda×density×v², where lambda=0.316/Reynolds number 0.25 and v²=square of mean velocity. Laminar flow is more “efficient” than turbulent flow. In laminar flow, P1−P2=mean velocity whereas in turbulent flow, P1−P2=mean velocity^{1.75} Schlichting, H., supra, pp. 502-506. Therefore, much kinetic energy is wasted in turbulent flow; if the blood flow in humans would be turbulent, higher arterial pressure would be necessary to perfuse distant organs.

The vortices and eddies of turbulent flow subject the endothelium to large multidirectional oscillatory stresses that cause functional and morphological changes and activation or destruction of the endothelial and circulating cells, and the release of inflammatory cytokines and oxidants. Very high turbulence and wall shear stress cause erosion, denudation or necrosis of the endothelium, and lysis of circulating cells. See, e.g., Summers, D. S., “Essential hemodynamic principles,” in Rutherford, R. B., Vascular Surgery, WV Saunders Co., 1994, pp. 18-11 and 411; Fry, D. L., “Acute vascular endothelial changes associated with increased blood velocity gradients,” Circ. Res., Vol. 22, pp. 165-97 (1968); Fung, Y. C. et al., “Elementary mechanics of the endothelium of blood vessels,” J. Biomech. Eng., Vol. 11, pp. 1-12 (1993); Dewey, C. F. Jr. et al., “The dynamic response of vascular endothelial cells to fluid shear stress,” J. Biomech. Eng., Vol. 103 pp. 177-85 (1981); Zarins, C. K. et al., “Carotid bifurcation atherosclerosis. Quantitative correlation of plaque localization with flow velocity profiles and wall shear stress,” Circ. Res., Vol. 54 (1983), pp. 502-14; Bassiouny, H. S. et al., “Anastomotic intimal hyperplasia: mechanical Injury or flow Induced,” J. Vas. Surg., Vol. 15, pp. 708-716 (1992); Stein, P. D. et al., “Hemorheology of turbulence,” Biorheology, Vol. 17, pp. 301-19 (1980); De Wachter, D. S. et al., “Red cell injury assessed in a numeric model of a peripheral dialysis needle,” ASAIO J., Vol. 42, pp. M524-9 (1996). In turbulent flow, cells behaves like missiles colliding at high velocity against the vessel wall or against other cells.

Any increase of the velocity of blood during dialysis, i.e. increasing the dialysis blood flow rate to achieve higher removal of urea, increases exponentially the turbulence (and turbulent shear stress) post venous needle because turbulence depends on the square of the fluctuating velocity unlike laminar flow in which the shear stress is proportional to the mean velocity of the flow.

Turbulent shear stress is proportional to the square of the fluctuating velocity. The segment of the vascular access downstream of the venous needle is the only vessel in the body with turbulent flow.

Cytokines are proteins produced by many cell types that modulate the function of other cells and play important roles in acute and chronic inflammation and humoral and cell mediated immunity. Cytokines can be divided into types of pro-inflammatory, i.e. IL-1, IL-6, TNF-a, immune-regulatory, i.e. IL-2, IL-12, and anti-inflammatory, i.e. IL-10.

Studies performed with SBIR Phase I Grants 1R43DK55385-01A1 and 1R44DK059062-02A1, and SBIR Phase II Grant 5R44DK059062-03 (2005-2009) have demonstrated the following: (1) blood exits the venous dialysis needle at a velocity of 3-7 m/s which is up to 100 times higher than the velocity of peripheral veins and causes high turbulence inside the dialysis access; (2) hemodialysis causes hemolysis of aged blood from a blood bank; (3) the high velocity/turbulence of the venous needle flow causes endothelial damage in sheep; (4) hemodialysis damages red cells, white cells, and platelets and causes oxidative stress in sheep; and (5) hemodialysis causes hemolysis and activation of platelets in humans.

Activation is stimulation of the cells to synthesize biological agents, usually by gene up-regulation or stimulation to release biological agents.

From Needle Study:

There are consequences to the damage to the endothelium and the release of cytokines and oxidants of cells. In particular, the damage to the endothelium and to circulating cells caused by the high velocity and turbulence of the flow past the venous needle during dialysis can cause or contribute to cause several complications in dialysis patients.

First, there can be vascular access complications. The high turbulence of the flow causes endothelial damage. Endothelial damage always heals as intimal hyperplasia, and intimal hyperplasia is the cause of “late” vascular access stenosis and thrombosis in most arterio-venous fistulas and grafts used as dialysis vascular accesses. Repetitive endothelial damage in the same site of the vascular access during four hours of dialysis every other day can lead to stenosis and thrombosis of the vascular access. Access complications, principally stenosis and thrombosis, are the major cause of morbidity of hemodialysis patients, they consume >10% of the total cost of care of all the dialysis patients, and the occurrence of access complications is reaching epidemic proportions. Schwab, S. J., “What Can Be Done to Preserve Vascular Access for Dialysis?,” Seminars in Dialysis, Vol. 14, pp. 152-153 (1991); Feldman, H. I. et al., “Hemodialysis vascular access morbidity in the United States,” Kidney Int., Vol. 43, pp. 1091-1096 (1993); Bleyer, A. J., “The cost of hospitalizations due to hemodialysis access management,” Nephrology News & Issues, Vol. 9(1), pp. 19-22 (1995); Pang-Yen and Schwab, S. J., “Vascular Access: concepts for the 1990s,” J. Am. Society Nephrology, Vol. 3, pp. 1-11 (1992); Schindler, R. et al., “Effect of the haemodialysis membrane on the inflammatory reaction in vivo,” Clin. Nephrol., Vol. 53, pp. 452-459 (2000). Also, failure of dialysis fistulas and grafts leads to the use of dialysis catheters that cause frequent infections, chronic inflammation and even death. At present, the cost of care of the vascular access is over $3B per year.

Second, there is a release of proinflammatory cytokines. In particular, pro-inflammatory cytokines IL-1, IL-6 and TNF-a are released from peripheral blood mononuclear cells (PBMC) and contribute to cause the inflammatory/hypercatabolic syndrome present in many dialysis patients that is manifested by anorexia, hypoalbuminemia, muscle wasting, and increased cardiovascular mortality and morbidity. Kaysen, G. A. et al., “Determinants of albumin concentration in hemodialysis patients,” Am. J. Kidney Dis., Vol. 29, pp. 658-668 (1997); Kaysen, G. A. et al., “Mechanism of hypoalbuminemia in hemodialysis patients,” Kidney Int., Vol. 48, pp. 510-516 (1995); Kaysen, G. A. et al., “The acute-phase response varies with time and predicts serum albumin levels in hemodialysis patients,” Kidney Int., Vol. 58, pp. 346-352 (2000); Qureshi, A. R. et al., “Inflammation, malnutrition, and cardiac disease as predictors of mortality in hemodialysis patients,” J. Am. Soc. Nephrol., Vol. 13, pp. S28-S36 (2002); Bergstrom, J. et al., “Elevated CRP is a strong predictor of increase mortality and low serum albumin in HD patients,” J. Am. Soc. Nephrol., Vol. 6, pp. 573 (1995); Bergstrom, J., “Nutrition and mortality in hemodialysis,” J. Am. Soc. Nephrol., Vol. 6, pp. 1329-1341; Iseki, K. et al., “CRP and risk of death in chronic dialysis patients,” Nephrol. Dial. Transplant, Vol. 14, pp. 1956-1960 (1999); Zimmerman, J. et al., “Inflammation enhances cardiovascular risk and mortality in hemodialysis patients,” Kidney Int., Vol. 55, pp. 648-658 (1999); Stenvinkel, P. et al., “Strong association between malnutrition, inflammation and atherosclerosis in chronic renal failure,” Kidney Int., Vol. 55, pp. 1899-1911 (1999); Danesh, J. et al., “Low grade inflammation enhances cardiovascular risk and mortality in HD patients,” B. M. J., Vol. 321, pp. 174-175 (2000); Stenvinkel, P., “Inflammation in end-stage renal failure: could it be treated?,” Nephrol. Dial. Transplant 17 Suppl 8:33-38 (2002); Amore, A. et al., “Immunological basis of inflammation in diaysis,” Nephrol. Dial. Transplant 17 Suppl 8: 16-24 (2002); Held, P. J., et al., “Mortality and duration of hemodialysis treatment,” JAMA, Vol. 265, pp. 871-875 (1991); Erkan, E. et al., “Role of Nitric oxide, endothelin-1 and inflammatory cytokines in blood pressure regulation in hemodialysis patients,” Am. J. Kidney Dis., Vol. 40(1), pp. 76-81 (2002); Foley, R. N. et al., “Impact of hypertension on cardiomyopahty morbidity and mortality in ESRD,” Kidney Int., Vol. 49, pp. 1379-1385 (1996); Sarnak, M. J. et al., “Epidemiology of cardiac disease in dialysis patients,” Seminars in dialysis, Vol. 12, pp. 69-76 (1999); Wanner, C. et al., “C-reactive protein a marker for all-cause and cardiovascular mortality in haemodialysis patients,” Nephrol. Dial. Transplant 17 [suppl 8]: 29-32 (2002); Bergstrom, J. et al., “What are the causes and consequences of the chronic inflammatory state in chronic dialysis patients?,” Seminars in Dialysis, Vol. 13(3), pp. 163-164 (2000); Stenvinkel, P. et al., “Strong association between malnutrition, inflammation and atherosclerosis in chronic renal failure,” Kidney Int., Vol. 55, pp. 1899-1911 (1999). These cytokines induce the systemic acute-phase inflammatory response associated with infection or injuries which include suppression of negative acute phase reactants (albumin, transferring, prealbumin), increase in positive acute phase reactants (CRP, fibrinogen, lipoprotein (a), sedimentation rate, haptoglobin and serum amyloid A). IL-1 and TNF-a stimulate the expression of IL-6, induce fever, anorexia and increase vascular permeability. IL-6 and TNF-a causes hypoalbuminemia by suppressing the hepatic synthesis of albumin, increase proteolysis in muscle and increases the hepatic synthesis of CRP protein. These cytokines also enhance leukocyte cytotoxicity and natural killer T cells which stimulate the release of other proinflammatory cytokines. The cytokines also stimulate endothelial cells to synthesize other cytokines, nitric oxide, and ET-1, to release ROS and the adhesion of circulating platelets and neutrophils. These cytokines are also released from activated platelets which also relocate p-selectin to the platelet surface. P-selectin mediates the adhesion of activated platelets to monocytes and neutrophils to form platelet-leucocytes complexes that also activate leucocytes.

Third, there is a release of reactive oxygen species (ROS). ROS are formed in mitochondria of monocytes, neutrophils, platelets and endothelial cells during aerobic metabolism by the reduction of oxygen to water. The major oxidants formed are superoxide, hydrogen peroxide and hydroxyl radicals. ROS toxicity is decreased by oxidation of lipids, proteins, peptides and nucleic acids. For example, ROS oxidize cholesterol in low density lipoprotein to form oxLDL, malondialdehyde (MDA), and 4-hydroxinonenal (HNE), proteins (principally albumin, measured as advanced oxidation protein products or AOPP), and peptides, reduced glutathione (GSH) is oxidized to glutathione disulfide (GSSG). Nitric oxide (NO) released from monocytes, neutrophils and endothelial cells combines with superoxide to decrease free oxygen radicals and form peroxynitrite and peroxynitric acid. Oxidants stimulate the release of proinflammatory cytokines and ET-1, promote adhesion of circulating cells to the endothelium and the activation of cells, and cause multiple cells dysfunctions. At high concentration, oxidants induce cell necrosis. Oxidative stress is believed to cause atherosclerosis, erythropoietin-resistant anemia, hypertension and myocardial dysfunction. See, e.g., Loughrey, C. M. et al., “Oxidative stress in hemodialysis,” W. J. Med., Vol. 87, pp. 679-683; Siems, W. et al., “Elevated serum concentration of cardiotoxic lipid peroxidation products in chronic renal failure in relation to severity of anemia,” Clin. Nephrol., Vol. 58-Suppl 1/2002(S20-S25); Roselaar, S. E. et al., “Detection of oxidants in uremic plasma by electron spin resonance spectroscopy,” Kidney Int., Vol. 48, pp. 199-206 (1995); Daschnrer, D. et al., “Influence of dialysis on plasma lipid peroxidation products and antioxidant levels,” Kidney Int., Vol. 50, pp. 1268-1272 (1996); Aiello, S. et al., “Nitric oxide/endothelin balance after nephron reduction,” Kid. Int., Vol. 53, Suppl 65, pp. S63-S67 (1998); Sakar, S. R. et al., “Nitric oxide and hemodialysis,” Seminars in Dialysis, Vol. 17(3), pp. 224-228 (2004); Bayes, B. et al., “Homocysteine, C-reactive protein, lipid peroxidation and mortality in haemodialysis patients,” Nephrol. Dial. Transplant, Vol. 18, pp. 106-112 (2003); Mezzano, D. et al., “Inflammation, not hyper-homocysteinemia, is related to oxidative stress and hemostatic and endothelial dysfunction in uremia,” Kidney Int., Vol. 60, pp. 1844-1850 (2001).

Many of these diseases contribute to cause the 20% mortality observed among dialysis patients. See, e.g., Renal Data System: 2003 Annual Data Report: Atlas of End-Stage Renal Disease in the United States. Am. J. Kidney Dis., Vol. 42, pp. S1-S230 (suppl 5) (2003).

Billions of cells circulate through the dialysis circuit during one dialysis and can be destroyed or activated if the velocity and turbulence of the blood is high. During a hemodialysis of 4 hours with a blood flow rate of 450 mL/min, about 108 L of blood circulates through the dialysis circuit. If the patient has a circulating blood volume of 4.0 L, all the circulating volume of blood and all the cells in the blood would circulate through the dialysis circuit every 9 minutes for 26 times, that is, about 650 million to 1.3 billion leucocytes, about 432 billion erythrocytes, and about 16 billion platelets. This gives ample opportunity to all the cells in the circulation and many cells recruited from the bone marrow to circulate through the dialysis circuit and become activated.

FIG. 9 shows the interactions between pro-inflammatory cytokines and other humoral factors when mononuclear cells are activated by high velocity and turbulence of the blood flow during hemodialysis. This unifying theory could explain the occurrence of many complications in dialysis patients. In particular, the activation of cells during dialysis stimulates the release of pro-inflammatory cytokines and other humoral factors that increase the magnitude of inflammation and oxidative stress. The inflammatory and oxidative processes started by dialysis persist several hours after dialysis is ended. Humoral factors released from circulating cells interact with endothelial cells. The activation of circulating leucocytes and the release of proinflammatory cytokines, principally IL-6, appear to be the primary factors to trigger inflammation and oxidation. Increased angiotensin II would also be a primary factor with rapid fluid removal. Rapid fluid removal also would increase catecholamines which further stimulate the release of angiotensin II, oxidants and ET-1 which would be indirectly pro-inflammatory because oxidants and ET-1 stimulate the release of pro-inflammatory cytokines.

Fundamental principles of fluid dynamics set the starting point from which the problem of damaged blood cells may be evaluated. See, e.g., Davies, J. T., Turbulence Phenomena, Academic Press (1972), pp. 1-153; Munson, B. R. et al., Eds., Fundamentals of Fluid Mechanics, New York, John Wiley & Sons. (1990), pp 484-547; Schlichting, H., Ed., Boundary Layer Theory, 4th Ed., New York, McGraw-Hill (1960), pp. 502-506; Fry, D. L., “Acute vascular endothelial changes associated with increased blood velocity gradients,” Cir. Res. 22:165-197 (1968).

In addition, a variety of fluid dynamics studies have been performed for simulated blood dialysis circuits, and hemodialysis studies have been performed in sheep and humans. See, e.g., Zarate, A. R., “New Needle for Two-Needle Hemodialysis,” ASAIO Journal, 44 (1998), M549-554; Zarate, A. R., “Venous dialysis needle,” U.S. Pat. No. 5,662,619, issued Sep. 2, 1997; Zarate, A. R., “Venous Device,” International Pub. No. WO 2009/005644, published Jan. 8, 2009; Zarate, A. R., “Vascular Graft Prosthesis,” U.S. Pat. No. 5,849,036, issued Dec. 15, 1998; Unnikrishnan, S., Zarate, A. R., Jones, S. A., and Anayiotos, A. S., “Hemodynamic Evaluation of a Novel Hemodialysis Needle,” Biomedical Engineering Society-Southern Conference, Nashville, September 2003; Zarate, A. R., Final report to NIH, grant 1R43DK55385-01A1, Mar. 25, 2004, “New needle for two-needle hemodialysis,” studies performed with Dr. Giddens at Georgia Tech and at U. Alabama Birmingham; Zarate, A. R. et al., “A Novel Venous Dialysis Needle for Two Needle Hemodialysis,” Georgetown University, Washington Hospital Center, George Washington Univ., Washington, D.C., Abstract, J. Am. Soc. Neph. 11, Toronto 2000, A1073; Zarate, A. R. et al., “The Current Venous Dialysis Needle Jet Damages the Endothelium,” published in the proceedings of and presented as at the Amer. Society of Nephrology meeting, Nov. 7, 2008, Philadelphia, Pa.; Zarate, A. R. et al., “A Novel Venous Dialysis Needle Causes Less Damage to the Endothelium than the Current Venous Needle,” Poster, International Hemodialysis Society meeting, Hong Kong, August 2009; Zarate, A. R. et al., “Fast Hemodialysis Damages Circulating Blood Cells,” Abstract published in the proceedings of the Amer. Society of Nephrology meeting, Nov. 7, 2008, Philadelphia, Pa.; Zarate, A. R. et al., “A Novel Venous Dialysis Needle is Safe and Effective in Patients,” Abstract published in the proceedings of and presented at the Amer. Society of Nephrology meeting, Nov. 7, 2008, Philadelphia, Pa.; Zarate, A. R. et al., “A Novel Venous Dialysis Needle Causes Less Damage to Circulating Blood Cells than the Current Venous Needle,” Abstract published in the proceedings of and presented at the Amer. Society of Nephrology meeting, Nov. 7, 2008, Philadelphia, Pa.; and Zarate, A. R. et al., “A Novel Venous Dialysis Needle Improves the Adequacy of Dialysis,” Poster presentation, World Congress Nephrology, May, 2009, Milan.

Such studies were performed with the support of several grants from the National Institute of Health. The fluid dynamic studies included flow visualization along with velocity and turbulence measurement by laser Doppler velocimetry in a model that simulated the hemodynamics of the flow past a venous needle. Those studies demonstrated, for example, that blood circulates in some segments of the current dialysis circuit at velocities up 100 times higher than the velocity of blood in peripheral veins, thus causing extremely high turbulence. In addition, the hemodialysis studies performed with stored human blood or performed in sheep and patients demonstrated that the circulation of blood through the dialysis circuit causes hemolysis, activation of circulating mononuclear cells and platelets, and an increase in the generation and release of oxidants.

With the benefit of the fundamentals of fluid dynamics and the above-identified research, the advances described herein have been made.

The various portions of Cheremisinoff, N. P., supra and Munson, B. R., supra cited throughout this disclosure, including the figures disclosed in those portions, are incorporated herein by reference thereto.

The designs of the distal (venous) end of current dialysis catheters present a variety of problems. FIGS. 30-32 show three major known designs of the distal end (or venous end) of double lumen dialysis catheters.

Turning first to FIG. 30, a tapered design, a dialysis catheter 300 has a distal end that is tapered and has one distal opening 304 and several other openings on the lateral size of the catheter close to the distal end 303. Blood exits through the distal end at very high velocity and through some of the lateral openings because of the high pressure at the distal end: when the large volume of blood reaches the tapered distal end, the progressively decreasing diameter increases the pressure and the high pressure forces some of the blood through the lateral openings. If the distal end would not be tapered, blood would not exit through the lateral orifices because blood coming at high velocity does not take sharp corners. This is a basic principle of fluid dynamics and has been demonstrated in previous studies. Blood exits the distal end at extremely high velocity because the distal end is tapered. For example, the distal end of some catheters has an internal diameter of 1.0 mm meaning that if 400 mL/min of blood were to exit this opening, it would do so at a calculated velocity of 7.1 m/s. In addition, in this design, the lateral openings through which blood enters the arterial catheter 301, the arterial catheter port 302 that connects with the arterial blood lines, the venous catheter port 305 that returns the cleaned blood and the direction of the blood flow inside the vein 306.

Turning next to FIG. 31, a staggered design, a dialysis catheter 310 has a venous or distal end 314 that is at least 1 cm longer than the distal end of the arterial catheter. Some designs have lateral openings 315. These lateral openings do not allow the exit of blood unless the pressure at the distal end is increased by occlusion against the wall or clots because, as mentioned above, blood running at high velocity does not take sharp corners. If the pressure is not increased, blood does not exit lateral openings. This design further includes an arterial port 318, a venous port 320, lateral openings of the arterial catheter 322, and the direction of the blood flow inside the vein is indicated by arrow 324.

As shown in FIG. 32, a split design, a dialysis catheter 330 includes a portion of 5-10 cm proximate an end of the catheter that is split in two catheters, (1) the arterial side and (2) the venous side which is 1-2 cm longer than the arterial side. The venous end of the catheter is shown 332. In some catheter designs of this type, there are lateral openings at the distal opening of the venous side 334. Again, blood does not exit these openings unless the pressure at the distal end is increased. This type of design further includes an arterial port 336, a venous port 338, and the direction of flow inside the vein where the catheter is inserted is shown by arrow 340.

### SUMMARY OF THE INVENTION

The dialysis blood circuit has been re-designed to decrease the velocity, turbulence and damage to circulating blood cells. Advantageously, such decreased damage to circulating blood cells in turn allows the realization of decreased incidence of medical complications and mortality caused by inflammation and oxidative stress, and concomitantly a decreased cost of care of dialysis patients.

In the circuit disclosed herein, the geometry of the current circuit has been changed to decrease the velocity, shear stress and turbulence of the blood flow. The velocity of the blood in some segments of the circuit is decreased by changing the shape or the internal diameter of several segments of the dialysis circuit and the turbulence is decreased by decreasing the velocity of the blood, eliminating irregularities protruding into the lumen of the circuit, and/or rounding corners or transitions of the circuit.

In one embodiment, a hemodialysis circuit includes a blood flow path between components including a cannula, fistula needle tubing, a blood line, and at least one selected from the group consisting of an artificial kidney and an air trap, wherein the blood flow path has no sharp transitions between the components. The hemodialysis circuit may further include at least one connector between two of the components, wherein the connector provides a smooth transition of the internal surfaces of the two components. In some embodiments, at least one free end of the connector may be tapered. The smooth transition may be provided by at least one internal arcuate surface of the two components. Also, the connector may be disposed between the fistula needle tubing and blood line. A free end of the blood line may have a larger internal diameter than an external diameter of a free end of the connector, and the free ends of the blood line and connector are directly coupled to one another. The artificial kidney may have a port and the blood line may be connected directly to the port. In addition, the cannula may be received within the fistula needle tubing. The hemodialysis circuit may include a blood pump. Further, the cannula may be formed of metal, or alternatively plastic or other materials.

In another embodiment, a system includes at least two components selected from the group consisting of an artificial kidney, an arterial blood line, fistula needle tubing, an arterial fistula needle, a venous blood line, an air trap chamber, a venous fistula needle. The at least two components are integrally formed. The arterial blood line may have an internal diameter of at least 3.0 mm or at least 4.0 mm. The venous blood line may have an internal diameter of at least 3.0 mm or at least 4.0 mm. A blood flow path may be formed between the at least two components and the blood flow path may have a diameter of at least 3.0 mm. Also, a blood flow path may be formed between the at least two components and the blood flow path may have a uniform diameter. The ratio of the internal diameters of two of the at least two components may be between 0.8 and 1.2.

In yet another embodiment, a system includes at least two components selected from the group consisting of an artificial kidney, an arterial blood line, fistula needle tubing, an arterial fistula needle, a venous blood line, an air trap chamber, a venous fistula needle. The at least two components are permanently coupled to one another. The arterial blood line may have an internal diameter of at least 3.0 mm or at least 4.0 mm. The venous blood line may have an internal diameter of at least 3.0 mm or at least 4.0 mm. A blood flow path may be formed between the at least two components and the blood flow path may have a diameter of at least 3.0 mm. Also, a blood flow path may be formed between the at least two components and the blood flow path may have a uniform diameter. The ratio of the internal diameters of two of the at least two components may be between 0.8 and 1.2.

A hemodialysis method may include: circulating blood from an arterial fistula needle through an artificial kidney and to a venous fistula needle, wherein the blood flows through a blood line and fistula needle tubing having the same internal diameter of at least 3.0 mm and without any change in diameter at an interface thereof. The blood may flow through a blood line and fistula needle tubing having the same internal diameter of at least 4.0 mm. A connector may be provided at the interface and the connector may couple the blood line and fistula needle tubing without any change in diameter therebetween. Also, the method may include pumping blood by compressing the blood line while blood is disposed therein, wherein the blood line has an internal diameter and is compressed to no less than 30% of the original diameter, no less than 50% of the original internal diameter, no less than 60% of the original internal diameter, or no less than 70% of the original internal diameter.

One embodiment of a venous dialysis catheter includes lateral openings and diverters inside the shaft located in the distal end of the catheter, with or without a tapered end. In some embodiments, the distal end is not beveled.

One embodiment of a double lumen dialysis catheter includes lateral openings and diverters inside the shaft of the venous catheter, located in the distal end of the venous catheter. In some embodiments, a tapered end may be provided while in others the end may be non-tapered.

Some embodiments of dialysis catheters include diverters, which may be of difference sizes and angles of inclination.

An exemplary method of flowing blood through a dialysis catheter may include reducing circulating cell damage by decreasing the velocity and turbulence of the flow.

Another exemplary method of flowing blood through a dialysis catheter may include reducing endothelial cell damage by decreasing the velocity and turbulence of the flow.

Yet another exemplary method of flowing blood through a dialysis catheter may include reducing velocity and turbulence.

### BRIEF DESCRIPTION OF THE DRAWINGS

Preferred features of the present invention are disclosed in the accompanying drawings, wherein:

FIG. 1 shows a cross-section of the arterial side of a dialysis blood circuit;

FIG. 2 shows a cross-section of the venous side of a dialysis blood circuit;

FIG. 3 shows a cross-section of a blood flow path through regions of differing diameters (reproduced from Cheremisinoff, N. P., supra, p. 54);

FIG. 4 shows another cross-section of a blood flow path through regions of differing diameters (reproduced from Munson, B. R., supra, p. 509);

FIG. 5 shows another cross-section of a blood flow path through regions of differing diameters (reproduced from Cheremisinoff, N. P., supra, p. 54);

FIG. 6 shows another cross-section of a blood flow path through regions of differing diameters (reproduced from Munson, B. R., supra, p. 509);

FIG. 7 shows another cross-section of a blood flow path through regions of differing diameters (reproduced from Munson, B. R., supra, p. 507);

FIG. 7A shows loss coefficient K_{L} as a function of R/D for flow in a 90° bend (reproduced from Munson, B. R., supra, p. 512);

FIG. 7B shows separated flow in a 90° bend (reproduced from Munson, B. R., supra, p. 512);

FIG. 8 shows a cross-section of a portion of a rotating blood pump;

FIG. 8B shows blood velocity as a function of the amount of compression created in the tubing in which the blood flows as a result of the blood pump rollers;

FIG. 9 shows the interaction between cytokines and other humoral factors;

FIG. 10 shows a cross-section of a connection between a metal cannula and fistula needle tubing;

FIG. 11 shows another cross-section of a connection between a metal cannula and fistula needle tubing;

FIG. 12 shows a cross-section of a connection between fistula needle tubing and an arterial blood line;

FIG. 13 shows another cross-section of a connection between fistula needle tubing and an arterial blood line;

FIG. 14 shows another cross-section of a connection between fistula needle tubing and an arterial blood line;

FIG. 15 shows another cross-section of a connection between fistula needle tubing and an arterial blood line;

FIG. 16 shows a cross-section of fistula needle tubing coupled to a blood line without a connector therebetween;

FIG. 17 shows another cross-section of fistula needle tubing coupled to a blood line without a connector therebetween;

FIG. 18 shows a cross-section of a connection between blood lines and an air trap;

FIG. 19 shows another cross-section of a connection between blood lines and an air trap;

FIG. 20 shows a cross-section of a connection between the blood line and the arterial and venous port of the artificial kidney;

FIG. 21 shows another cross-section of a connection between the blood line and the arterial and venous port of the artificial kidney;

FIG. 22 shows a cross-section of an artificial kidney and blood line formed of unitary construction;

FIG. 23 shows a cross-section of the rounding of the entrance of fluid in the blood circuit (reproduced from Munson, B. R., supra, pp. 507-508);

FIG. 24 shows another cross-section of the rounding of the entrance of fluid in the blood circuit (reproduced from Munson, B. R., supra, pp. 507-508);

FIG. 25 shows another cross-section of the rounding of the entrance of fluid in the blood circuit (reproduced from Munson, B. R., supra, pp. 507-508);

FIG. 26 shows a cross-section of the rounding of a region of an artificial kidney; and

FIG. 27 shows a cross-section of an exemplary connection between two components;

FIG. 28 shows a cross-section of another exemplary connection between two components;

FIG. 29 shows a cross-section of yet another exemplary connection between two components;

FIG. 30 shows a cross-section of a tapered design of a dialysis catheter;

FIG. 31 shows a cross-section of a staggered design of a dialysis catheter;

FIG. 32 shows a cross-section of a split design of a dialysis catheter;

FIG. 33 shows a cross-section of a single lumen, venous dialysis catheter;

FIG. 34 shows a cross-section of a catheter with lateral openings and diverters;

FIG. 35 shows a cross-section of use of a trocar with pointed tip (reproduced from WO 2009/005644);

FIG. 36 shows a cross-section of a dialysis catheter with a diverter;

FIG. 37 shows a cross-section of a dialysis catheter with bifurcation proximate an end thereof;

FIG. 38 shows a cross-section of the distal end of a dialysis catheter with an inverted funnel shape;

FIG. 39 shows a side view of a dialysis catheter with a lateral opening have a U shape (reproduced from WO 2009/005644);

FIG. 40 shows a side view of a dialysis catheter with a lateral opening have an oval shape (reproduced from U.S. Pat. No. 5,662,619);

FIG. 41 shows a side view of a dialysis catheter with a lateral opening have an circular shape (reproduced from U.S. Pat. No. 5,662,619);

FIG. 42 shows a side view of a dialysis catheter with a lateral opening have an rectangular shape (reproduced from U.S. Pat. No. 5,662,619);

FIG. 43 shows a side view of a dialysis catheter with a lateral opening have an parallelogram shape (reproduced from U.S. Pat. No. 5,662,619);

FIG. 44 shows a cross-section of a dialysis catheter with diverters (reproduced from WO 2009/005644);

FIG. 45 shows a cross-section of a dialysis catheter with lateral openings;

FIG. 46 shows a cross-section of a connector in a dialysis catheter;

FIG. 47 shows a cross-section of another connector in a dialysis catheter;

FIG. 48 shows a cross-section of yet another connector in a dialysis catheter;

FIG. 49 shows a cross-section of a dialysis catheter with lateral orifices at a venous end; and

FIG. 50 shows a cross-section of an air trap.

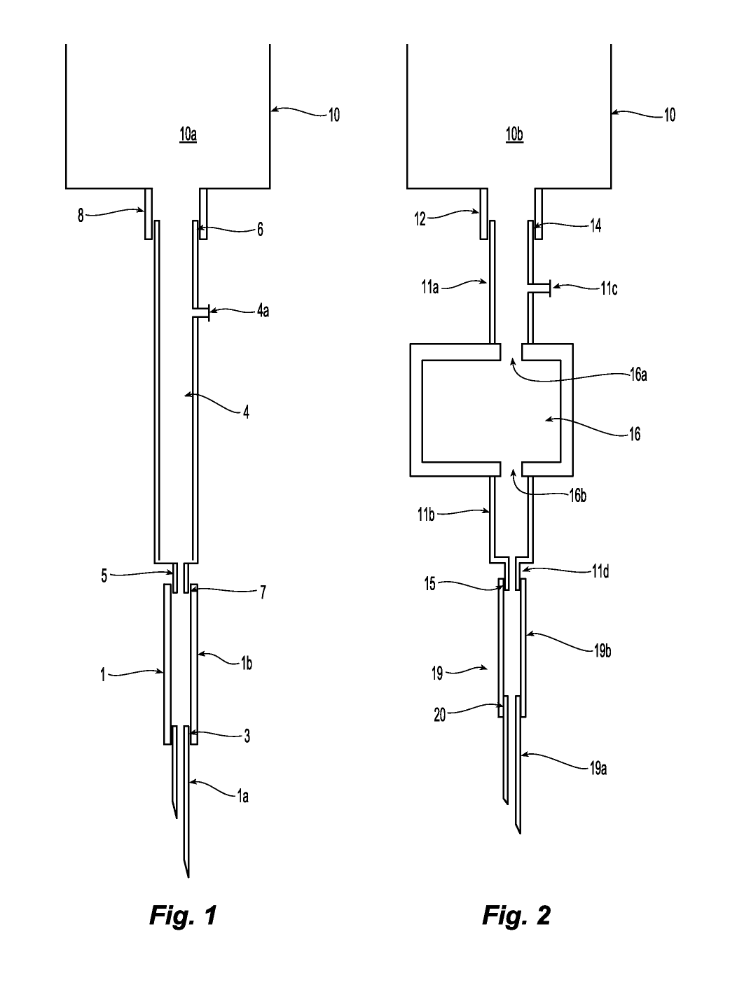

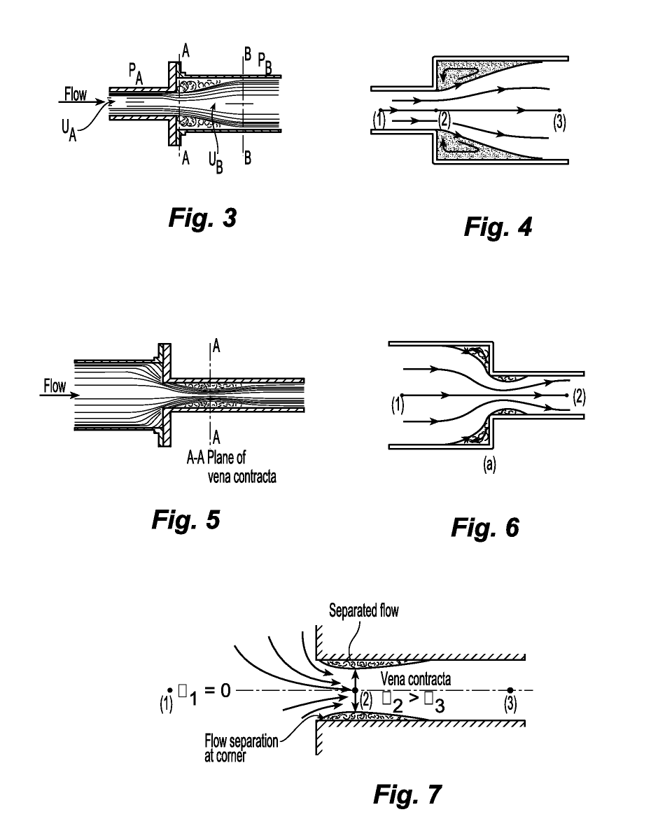

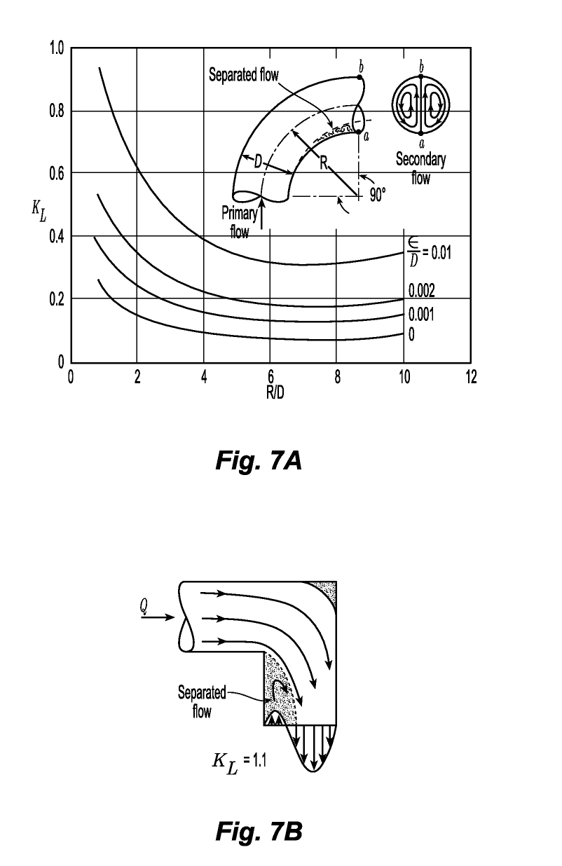

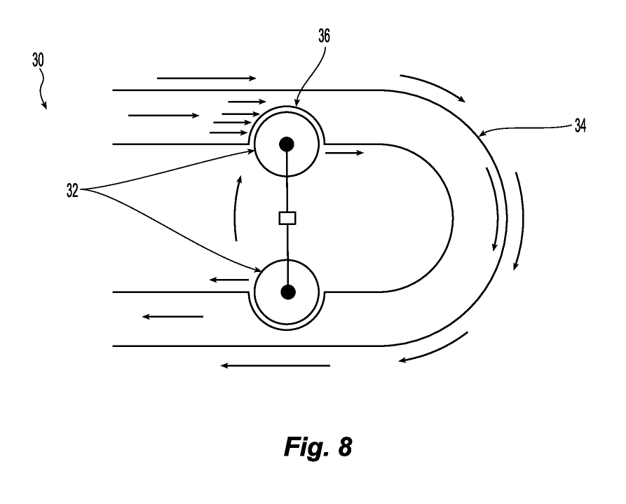

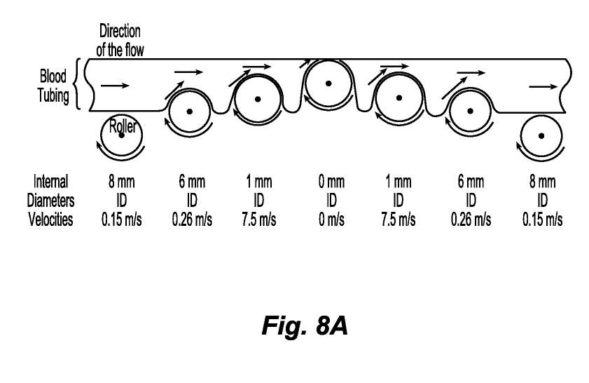

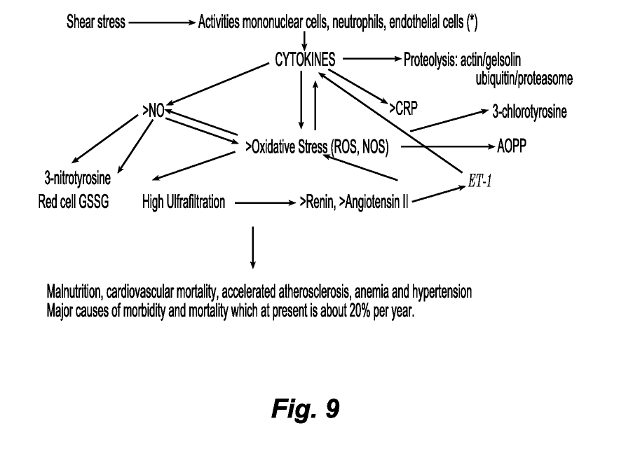

### DETAILED DESCRIPTION OF THE PREFERRED EMBODIMENTS

Material for the venous blood line. The ideal circuit should be manufactured with an elastic material that will allow expansion during the compression cycle and spontaneous contraction to regain initial diameter during the decompressing cycle.

A variety of connections between components of the dialysis circuit are contemplated to permit lower velocity and turbulence therein.

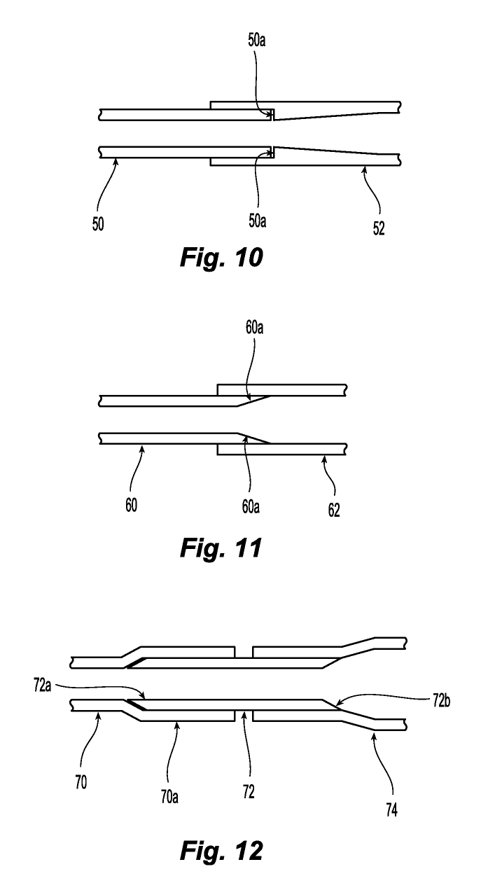

Connection between metal cannula and fistula needle tubing. In one embodiment, shown in FIG. 10, a free end of metal cannula 50 has a rim 50a that is “hidden” by plastic fistula needle tubing 52 proximate a free end thereof so that rim 50a does nor protrude into the lumen of tubing 52.

In another embodiment, shown in FIG. 11, a free end of metal cannula 60 has a tapered rim 60a such that the rim does not abruptly cause a change in diameter of the flow path when cannula 60 is connected with fistula needle tubing 62, but instead there is a gradual change in diameter.

Such designs of FIGS. 10-11 permit a decreased flow separation caused by an otherwise sudden increase in diameter. Also, the designs permit a decrease in the resistance to the flow and the pressure inside the dialysis circuit caused by the changes in diameter and flow separation.

Connection between fistula needle tubing and arterial blood line. Referring next to FIG. 12, fistula needle tubing 70 is provided with a flared free end 70a to receive a connector 72 with an optionally tapered first free end 72a. In addition, arterial blood line 74 has a contoured shape such that a second free end 72b of connector 72 fits in a free end thereof, and second free end 72b may be tapered to provide a non-abrupt transition between the internal diameter of connector 72 and the internal diameter of arterial blood line 74. Such a design permits decreased velocity and turbulence and decreased damage to circulating cells because there is no impact of circulating cells with the rim of connector 72.

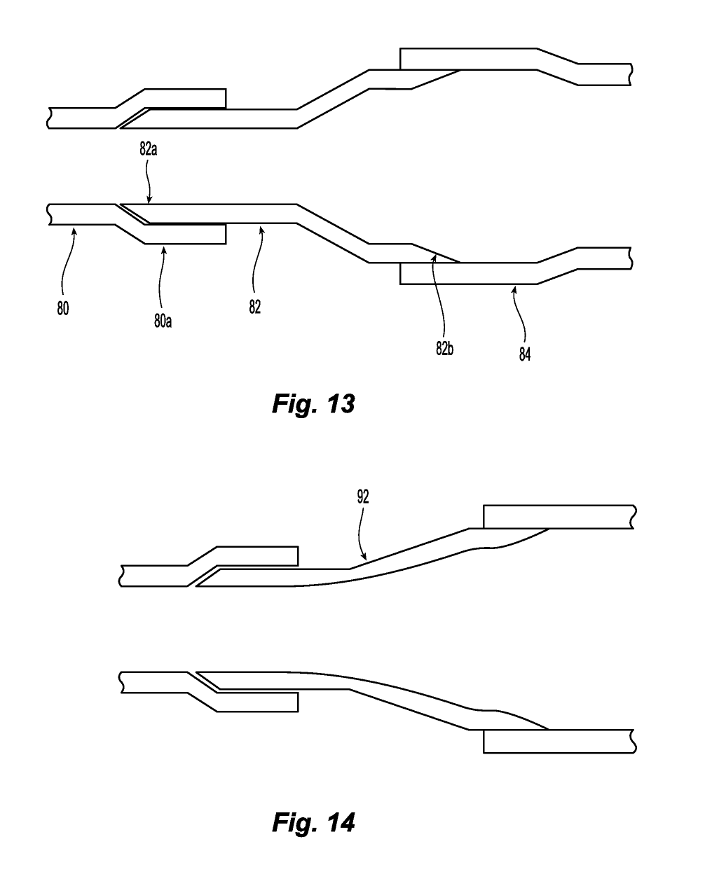

In another embodiment shown in FIG. 13, fistula needle tubing 80 is provided with a flared free end 80a to receive a connector 82 with an optionally tapered first free end 82a. In addition, arterial blood line 84 has a different contoured shape such that a second free end 82b of connector 82 fits in a free end thereof, and second free end 82b may be tapered to provide a non-abrupt transition between the internal diameter of connector 82 and the internal diameter of arterial blood line 84. Such a design permits decreased velocity and turbulence and decreased damage to circulating cells because there is no impact of circulating cells with the rim of connector 72.

In exemplary embodiments, the internal diameter of fistula needle tubing 70, 80 proximate a free end thereof is increased to 4-5 mm as shown in FIGS. 12-13 to permit connector 72, 82 to fit into fistula needle tubing 70, 80, while the internal diameter of the fistula needle tubing remote from the free end thereof and the internal diameter of the connector proximate the adjacent free end thereof are the same, e.g., 3 mm. In such a design, the ratio of internal diameter of the fistula needle tubing to the internal diameter of the connector is approximately 3:3, and the ratio of velocities is also 1 (at a flow rate of 600 mL/min., the calculated mean velocity is approximately 1.4 m/s). In such a design, the ratio of internal diameters is 3 mm to 2 mm, or 1.5, the ratio of velocities also is approximately 1.5, and the calculated mean velocity is 3.1 m/s. In other words, blood flowing in a connector with an internal diameter of 3 mm, disposed between the blood line and the fistula needle tubing, would have a flow rate of 600 mL/min. and a velocity of 1.4 m/s. Likewise, blood flowing in a blood line or fistula needle tubing with an internal diameter of 3 mm would have a flow rate of 600 mL/min. and a velocity of 1.4 m/s.

In some exemplary embodiments, the ratio of the internal diameter of one component of the dialysis system (e.g., selected from a blood line, fistula needle tubing, and a connector coupled thereto) to the internal diameter of another component of the dialysis system (e.g., selected from a blood line, fistula needle tubing, and a connector coupled thereto) is between 0.8 and 1.2.

In exemplary embodiments, the portion of the connector proximate a free end thereof that connects with the fistula needle tubing has the same internal diameter as the fistula needle tubing (e.g., 3 mm) and the rim of the connector does not protrude into the lumen of the fistula needle tubing because it is “hidden” by the plastic tubing. In such a design, the ratio of internal diameter of the fistula needle tubing to the internal diameter of the connector is approximately 3:3, the ratio of velocities also is 1 (at a flow rate of 600 mL/min. the calculated mean velocity is approximately 1.4 m/s). In such a design, the ratios of internal diameters and velocities are as mentioned above.

Still further, in exemplary embodiments, the end of the connector that connects with the arterial blood line either has the same internal diameter as the rest of the connector (e.g., 3 mm) and the blood line is compressed to adapt to a connector of smaller internal diameter, or the internal diameter of the connector is similar to the internal diameter of the blood line (e.g., 4 mm). In such a case, the end of the blood line is expanded to fit over the connector. In such a design, the ratio of internal diameters of the connector and blood line is approximately 4 mm to 4 mm, or 1, and the ratio of velocities is also 1 (at a flow rate of 600 mL/min. the calculated mean velocity is 0.8 m/s). In one exemplary design, the ratio of internal diameters is 2 mm to 4 mm, or 0.5, the ratio of velocities is similar, and the calculated mean velocity at a 600 mL/min. flow rate is 3.1 m/s.

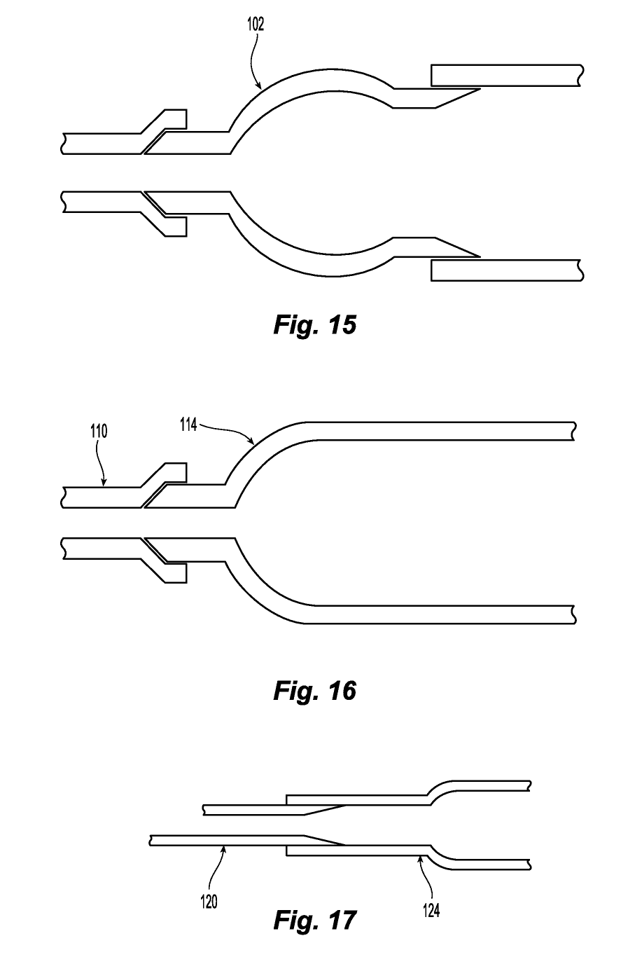

Alternate connector contours, e.g., internal and exterior shapes, are shown for example in connectors 92, 102, 112 of FIGS. 14-15, respectively.

In yet other embodiments, shown in FIGS. 16-17, fistula needle tubing 110, 120 is coupled to a blood line 114, 124 (e.g., arterial or venous) without a connector therebetween, thus providing a seamless connection. In such a design, decreases may be realized in the velocity and turbulence of the blood as well as damage to circulating cells.

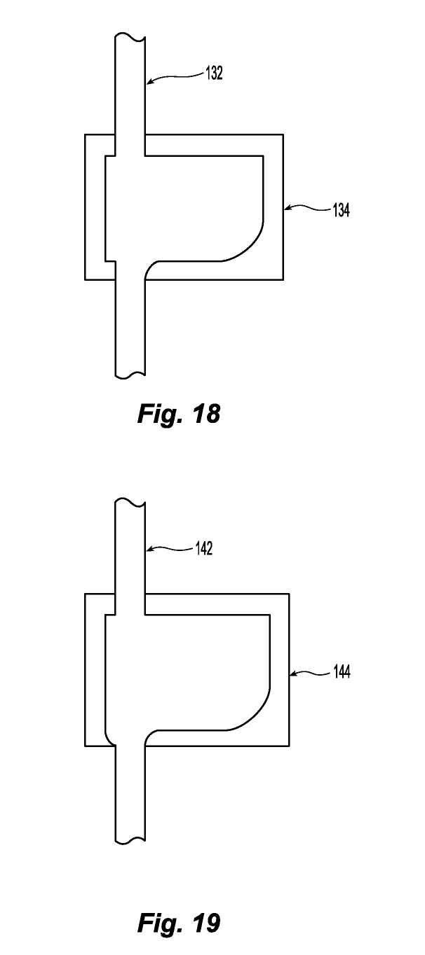

Turning to FIGS. 18-19, embodiments of connections between blood lines 132, 142 and the respective air traps 134, 144 are shown. In these embodiments, the connections have the same internal diameter as the blood lines (e.g., 4 mm). Such designs decrease the velocity and turbulence of the blood entering or exiting the air traps. In such designs, the ratio of internal diameters of the blood line to the connection is approximately 1 and the ratio of velocities is also approximately 1.

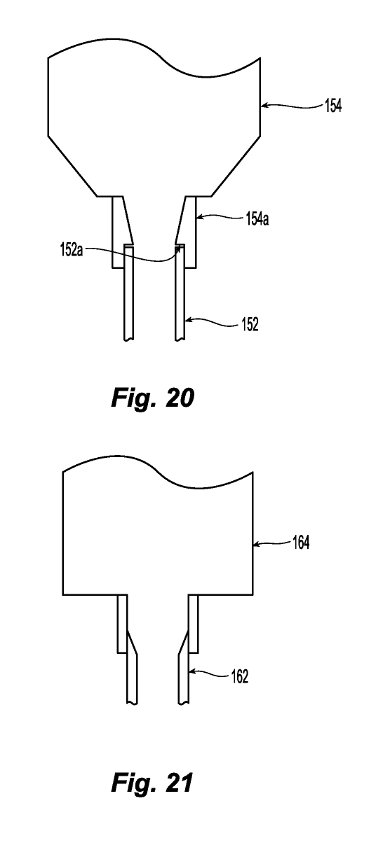

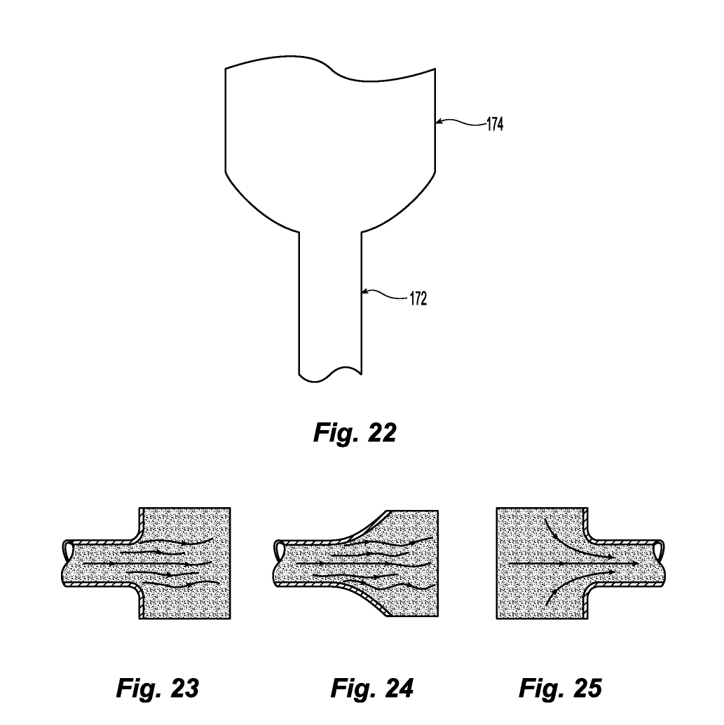

Referring next to FIGS. 20-21, embodiments of connections between the blood line and the arterial and venous port of the artificial kidney are shown. In the embodiment of FIG. 20, an artificial kidney 154 is provided with a port 154a such that the rim 152a of blood line 152 does not protrude into the lumen of the circuit. In the embodiment of FIG. 21, the end of blood line 162 is tapered so that there is a non-abrupt transition with artificial kidney 164 into the lumen of the circuit. As shown in the embodiment of FIG. 22, artificial kidney 174 and blood line 172 (e.g., arterial and/or venous) are formed of unitary construction and thus their connection is seamless. Such designs of the connection between the blood lines and the arterial and venous ports of the artificial kidney permit a decrease in the velocity and turbulence caused by changes in the internal diameter and also a decrease in the impact of cells against the rim of the blood lines.

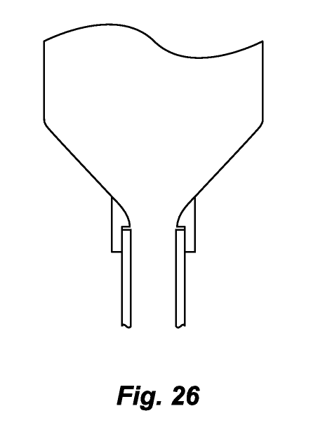

In some embodiments, the entrances and exits in the dialysis circuit tubing have been streamlined by providing arcuate transitions. For example, as shown in FIGS. 23-25 (from Munson, B. R., supra, pp. 507-508), rounding the entrance and exit of fluids in the blood circuit decreases turbulence or vortex formation. In such designs, rounded corners decrease the flow separation otherwise caused by sharp corners and also decrease the turbulence and cell damage. Such rounding of edges or corners also may be applied to the air traps as shown in FIGS. 18-19, the arterial and venous end of an artificial kidney shown in FIG. 26, and blood lines shown in FIG. 14.

In other embodiments, the connection between the arterial and venous ports of the dialysis catheter and the arterial and venous blood lines are streamlined. In such designs, the arterial and venous ports of the dialysis catheter are modified consistent with the designs as discussed above, and the interior of the connector may have a as previously described.

Advantageously, each of the aforementioned embodiments permits a decrease in the velocity and turbulence of the blood flow in the circuit and a decrease in the damage to circulating cells.

In some embodiments, the metal cannula may be replaced by a hard consistency polymer. If the metal cannula is replaced by a cannula of the same geometry and dimension but made of a hard consistency polymer, the polymer and the plastic tubing optionally could become one single unit without connectors.

Blood pumps. Any decrease in the tube diameter increases the velocity proportionally and causes turbulence. In turbulent flow, the turbulence is proportional to the square of the fluctuating velocity, that is, any decrease in diameter increases the velocity proportionally and increases the turbulence exponentially. In order to deliver a pre-set blood flow rate with fewer changes in velocity and turbulence (from compression cycle to decompression cycle), the blood pump preferably should have more than two rollers, e.g., three to six rollers (for example, three rollers for a pump that otherwise currently has two rollers), compression of the tubing preferably should not be more than 80% of the internal diameter, and more preferably not more than 50-60% of the internal diameter, and the frequency of compression preferably should be increased. This would be in contrast to some current dialysis blood pumps that have two rollers which rotate at 80 revolutions/min. to deliver a blood flow rate of 400 ml/min., with the rollers causing complete occlusion of the tubing during each cycle at a given time of the cycle. Ideally, if the pump would have many rollers and the compression of the tubing would be 50% or less, the flow could resemble a steady flow and not pulsatile flow and this would decrease velocity, turbulence, and damage to circulating blood cells.

In laminar flow, the velocity and shear stress increase are proportional to the increase in blood flow rate or decrease in the diameter of the tubing. In contrast, in flow that occurs inside the tubing when it is almost fully collapsed by the roller, the flow is turbulent and the velocity increase is proportional to the fluctuating velocity. It would be less harmful to circulating cells if the blood pump would have more than two rollers (e.g., three) and the rollers would cause no more than an 80% decrease in the internal diameter of the tube, preferably no more than a 60% decrease in the internal diameter of the tube, and for example no more than a 50% decrease in the internal diameter of the tube.

For example, in comparing the velocity of blood with various degrees of compression of the tubing by the blood pump rollers, an internal tubing diameter of 8 mm would result in a velocity of 0.15 m/s, an internal tubing diameter of 6 mm would result in a velocity of 0.26 m/s, an internal tubing diameter of 1 mm would result in a velocity of 7.5 m/s, and an internal tubing diameter of 0 mm would result in a velocity of 0 m/s.

To decrease friction of the roller against a short segment of the tubing, the head of the pump could move forward up to 1 inch during the entire duration of dialysis, so that the rollers contact a longer segment of the tubing. This will decrease the potential for the roller to damage the tube.

All the above embodiments can be used for hemodialysis, to transport blood in other settings, e.g., extracorporeal perfusion during cardiac bypass surgery, or any other fluids when there is a need to decrease the velocity and or turbulence of the fluid.

Discussion herein with respect to embodiments with arterial blood lines apply likewise to venous blood lines.

Integrally Formed or Permanently Coupled Components. In some embodiments, various components of the hemodialysis circuit are integrally formed or permanently coupled to one another. Examples include: (1) an artificial kidney may be integrally formed or permanently coupled to an arterial blood line, the arterial blood line may be integrally formed or permanently coupled to fistula needle tubing, and/or the fistula needle tubing may be integrally formed or permanently coupled to an arterial fistula needle; and (2) an artificial kidney may be integrally formed or permanently coupled to a venous blood line, the venous blood line may be integrally formed or permanently coupled to an air trap chamber, the venous blood line may be integrally formed or permanently coupled to fistula needle tubing, and/or the fistula needle tubing may be integrally formed or permanently coupled to a venous fistula needle. Advantageously, in such constructions, the use of connectors between two or more of the components may be obviated such that sharp transitions, for example, may be avoided between the various portions of the dialysis circuit.

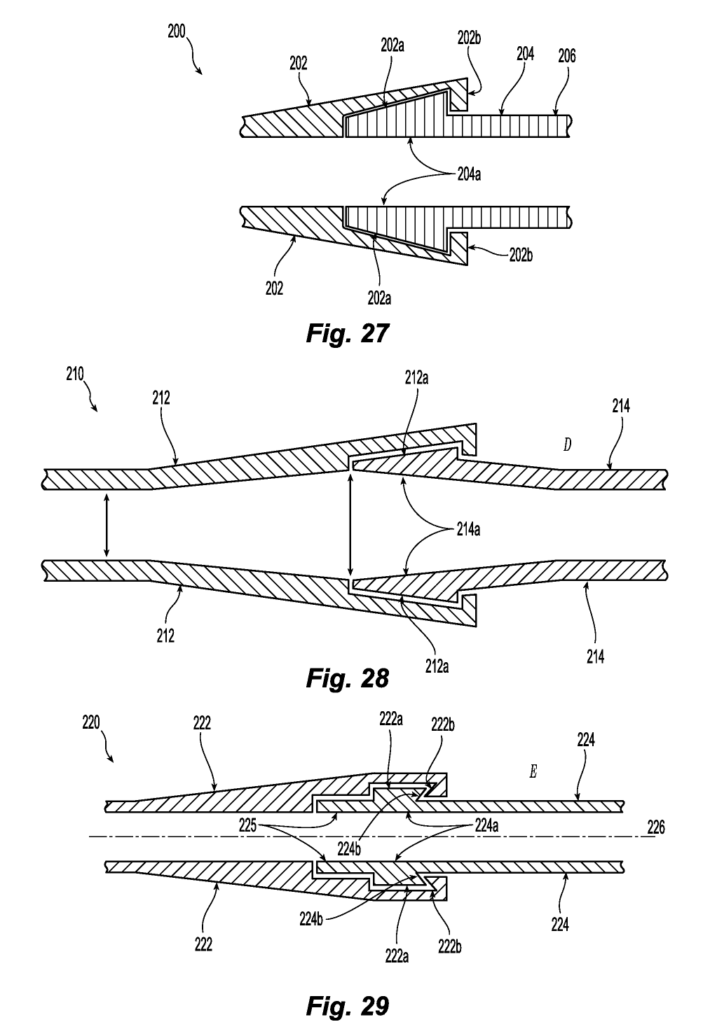

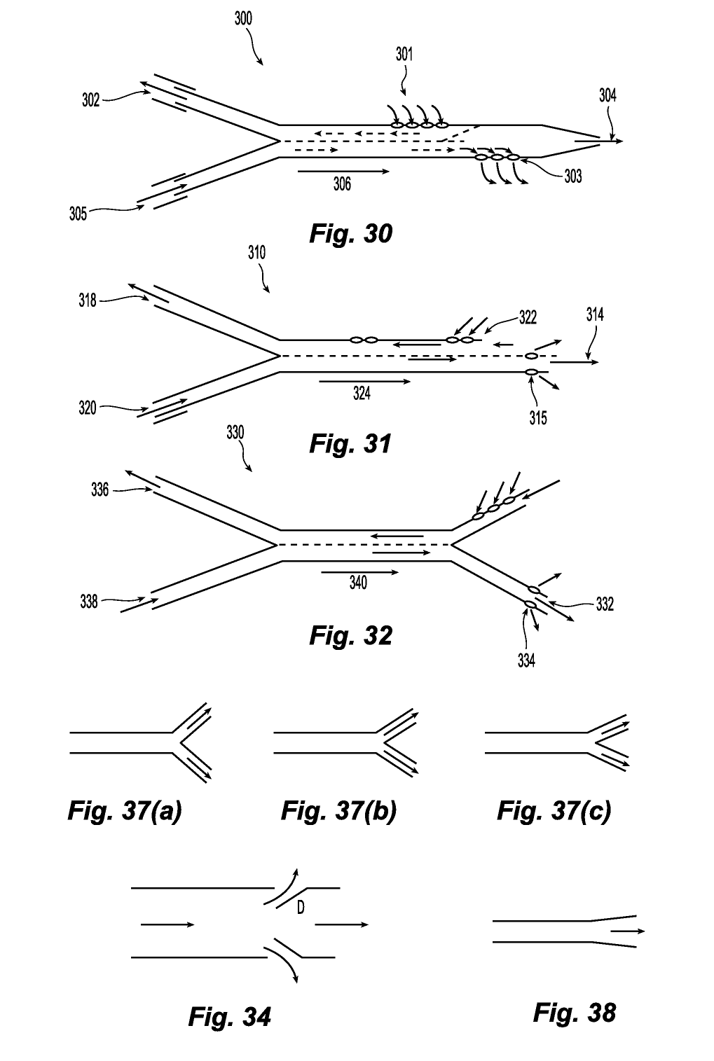

Connections. Turning to FIGS. 27-29, exemplary connections are shown for providing smooth transitions between components. Such connections, for example, may find use outside the field of dialysis. As shown in FIG. 27, connection 200 is formed between components 202, 204. Component 202 includes a socket 202a while component 204 includes a head 204a. When making a connection to “lock” the components to each other, head 204a snap-fits within socket 202a, with socket 202a including a free end 202b disposed to resist removal of head 204a from socket 202a. To “unlock” components 202, 204 (which may be formed, for example, from flexible or semi-rigid polymer tubing) from each other, the outer diameter of component 204 may be compressed such as proximate a region 206 adjacent head 204a, thus permitting head 204a to be removed from socket 202a. In some embodiments, socket 202a and head 204a extend radially about the entire circumference of the respective components. In other embodiments, several sockets and heads instead may be provided radially about the circumference of the respective components, such as being separated by 180° or 120°. Turning to FIG. 28, connection 210 is similar to connection 200 as described above except that a region of gradually increased internal diameter is provided between components 212, 214 proximate socket 212a and head 214a. Such a design permits the connection of components without decreasing the internal diameter and without protrusions that can cause turbulence and damage circulating cells during dialysis. And, as shown in FIG. 29, in another exemplary embodiment, connection 220 is provided between components 222, 224. Socket 222a and head 224a are provided with surfaces to facilitate coupling, including opposing surfaces 222b, 224b disposed parallel to one another and transverse but not perpendicular to longitudinal axis 226, as well as extension 225 of head 224a fitting in a like-sized portion of socket 222a.

In some embodiments, permanent coupling of components may be achieved, for example, using a high strength adhesive or otherwise bonding the components together such as with welding.

The presence of connectors between components of the dialysis circuit permits connection and disconnection of the various components. However, in another exemplary embodiment, two or more components of the circuit are formed of unitary construction or permanently coupled to one another (for example, the port of the dialysis filter and the blood lines, or the blood lines and the dialysis fistula needle).

It will be appreciated that in the prior art, connections (such as made by connectors) between components of the hemodialysis circuit were standardized in size to have an internal diameter of about 2 mm and it was not recognized that changes in the internal diameter in the circuit could present significant and deleterious effects. Such standardization can be traced to the use of lines for blood transfusions or for the infusion of saline solution, which tubing had in internal diameter of 2 mm. In these cases, the fluids (blood or saline) run through the circuit at extremely low velocity and run only once (single pass), whereas in the dialysis circuit blood runs at extremely high velocity and about 96 L of blood circulates through the circuit—in other words, all the blood in the body circulates through the dialysis circuit about 27 times.

The use of larger diameter connections (such as made by connectors) between components of the dialysis circuit was not previously recognized as significant because of the lack of unawareness about the effects associated with high velocity blood flow, the circulation of blood many times through the dialysis circuit, and the damage caused to circulating blood cells by the high velocity and turbulence of the flow. The dialysis circuit disclosed herein, however, contemplates the use of connectors (connections) with a larger internal diameter (e.g., about 3 mm) for use at least in part, in decreasing the relative velocity, turbulence and damage to circulating blood cells as compared to the connections (such as made by connectors) of smaller internal diameter.

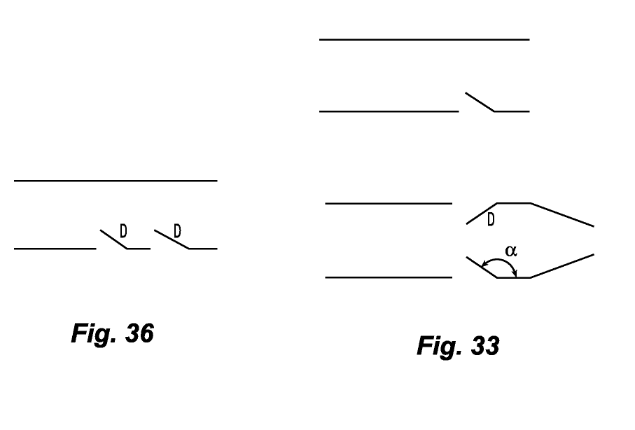

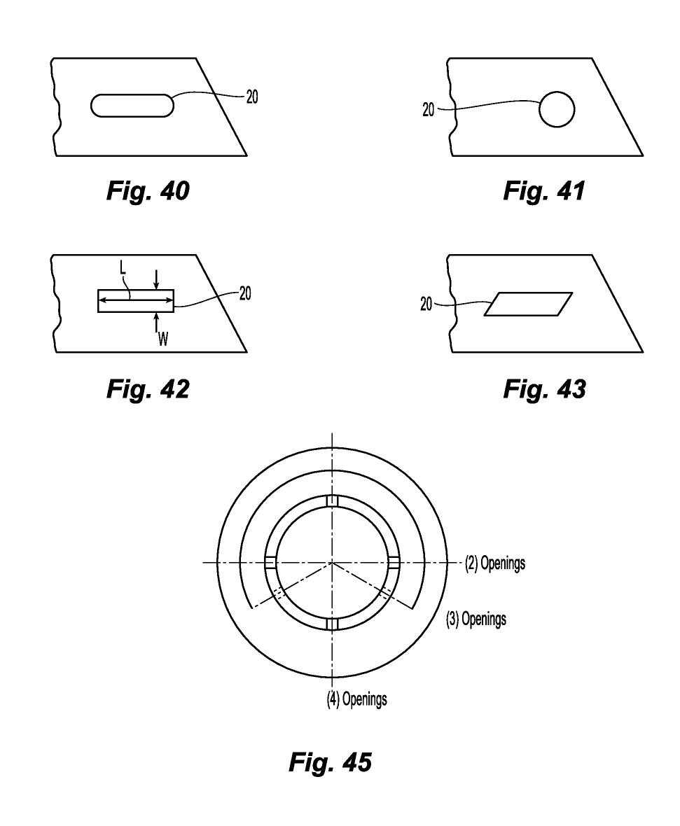

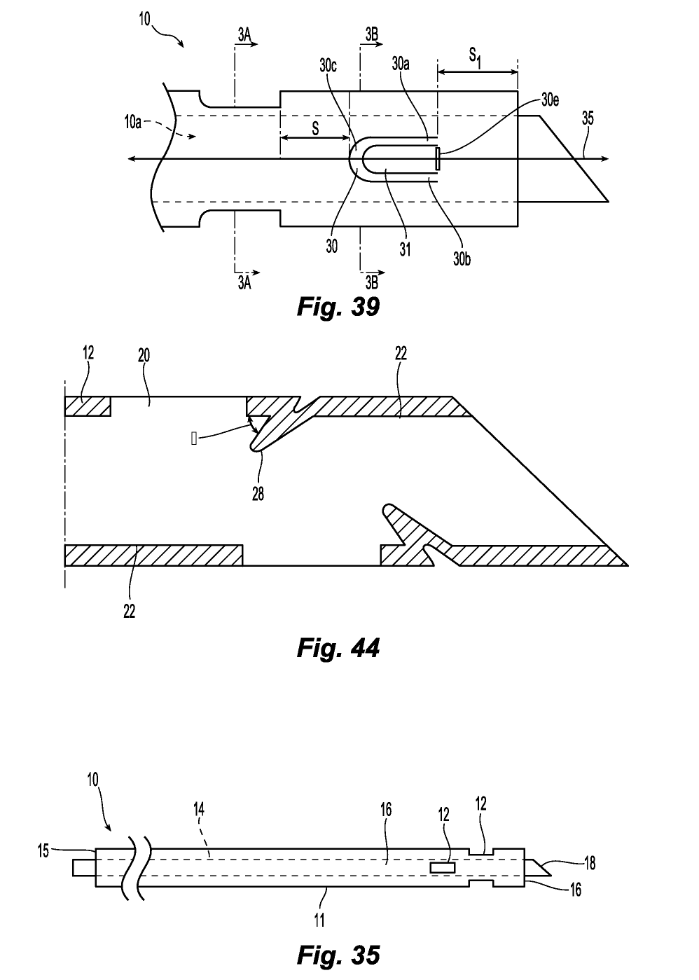

A variety of designs of dialysis catheters are contemplated, particularly as relates to the venous end thereof. In one exemplary embodiment of a single lumen, venous dialysis catheter, shown in FIG. 33, the distal end is tapered and has at least two lateral orifices with one diverter located inside of the lumen and in close proximity to the lateral openings. In some embodiments, angle α may be 30°. For various features that may be incorporated, see, e.g., U.S. Pat. No. 5,662,619 entitled “Venous Dialysis Needle” as well as U.S. Provisional Application No. 61/101,873 entitled “Method of improving fluid delivery.” As shown in FIG. 34, the distal end may not be tapered, the catheter may have two lateral openings with diverters, and it may be inserted with a trocar with pointed tip as shown for example in FIG. 35 as reproduced from WO 2009/005644.

In an exemplary embodiment of a double lumen, venous dialysis catheter, the distal end has at least two lateral orifices (FIGS. 34 and 36) with one diverter located inside of the lumen and in close proximity to the lateral openings. For various features that may be incorporated, see, e.g., U.S. Pat. No. 5,662,619 entitled “Venous Dialysis Needle,” U.S. Provisional Application No. 61/101,873 entitled “Method of improving fluid delivery,” Patent Cooperation Treaty International Application No. PCT/US08/07866 entitled “Method of reducing cell damage,” and U.S. Provisional Application No. 61/101,873 entitled “Method of improving fluid delivery.”

In the exemplary embodiment of a dialysis catheter of FIGS. 37(a)-(c), the distal end has no lateral opening but it bifurcates in two catheters (Y form). The angle of separation may be between 0% and 30%.

In another exemplary embodiment of a dialysis catheter, shown in FIG. 38, the distal end may have an inverted funnel shape.

In general, the distal end (or venous end) of single and double lumen dialysis catheters has been re-designed to decrease the velocity and turbulence of the blood exiting the catheter with the purpose of decreasing the damage to circulating blood cells and to the endothelium of the vein in which the catheter has been introduced. Advantageously, such decreased damage to circulating blood cells in turn allows the realization of decreased incidence of medical complications and mortality caused by inflammation, oxidative stress and damage or thrombosis of veins, and concomitantly a decreased cost of care of dialysis patients.

Prior art dialysis catheters include a tip or end of the venous catheter having a lateral opening disposed on a surface thereof to improve the flow of fluid out of the catheter. However, a lateral opening alone is ineffective because typical fluid dynamics result in fluid flow from the proximal end of the catheter to the opening at the distal end without desired flow through the lateral opening because fluids do not take sharp corners. In catheters with tapered distal ends, flow from a lateral opening may occur because the progressively smaller diameter of the tapered catheter increases the pressure inside the catheter which favors the exit of blood through the lateral opening. However, blood exits a distal opening of smaller diameter at much higher velocity, causing much higher turbulence. In catheters with complete or partial obstruction of the distal end (e.g., those attached to the vein wall or occluded by a clot), blood may exit through the lateral orifices.

Dialysis catheters as disclosed herein may have one or more lateral orifices on the surface of the venous catheter, close to the tip of the catheter and one diverter inside the catheter shaft close to each lateral opening.

Disclosures, including drawings, incorporated herein by reference include those in WO 2009/005644, U.S. Provisional Application No. 60/947,042 filed Jun. 29, 2007, WO 2009/005644 A2, PCT International Application No. PCT/US08/07866, U.S. Provisional Application No. 61/101,873, and U.S. Pat. No. 5,662,619.

In some preferred exemplary embodiments of dialysis catheters, the lateral openings have a U shape as shown for example in FIG. 39 reproduced from WO 2009/005644, but also could be other shapes such as oval or rectangular as shown for example in FIGS. 40-43 reproduced from U.S. Pat. No. 5,662,619.

In some preferred exemplary embodiments of dialysis catheters, two lateral openings are provided, but in other exemplary embodiments one or more lateral openings are provided.

In some exemplary embodiments of dialysis catheters, such as in a catheter of Fr 16 size, the lateral opening may have a length of 1.5 mm±0.2 mm and a height of 1.2 mm±1.0 mm. In catheters of smaller diameter, the length and height of the lateral opening may be decreased proportionally as shown for example in FIG. 39.

In some exemplary embodiments of dialysis catheters, the diverters in FIGS. 33, 34, 36, 39 may have a length of 0.7 mm±0.2 mm and a width of 1.0 mm±0.2 mm. Diverters could also be in the form shown in FIG. 44 reproduced from WO 2009/005644.

In some exemplary embodiments of dialysis catheters, the angle of inclination of the diverter D shown in FIGS. 33, 34, 36 may be about 30°, but this angle in some embodiments may be between about 25° and about 35°.

In some exemplary embodiments of dialysis catheters, the lateral openings for example may be located at 3 and 6 o'clock, or 9 and 12 o'clock, or if three lateral openings are provided at 4, 8 and 12 o'clock, as shown for example in FIG. 45 reproduced from U.S. Pat. No. 5,662,619.

In some exemplary embodiments of dialysis catheters, the distance of lateral opening from the distal end of the catheter preferably is 3-6 mm.

In some exemplary embodiments of dialysis catheters, the catheters do not have a beveled end.

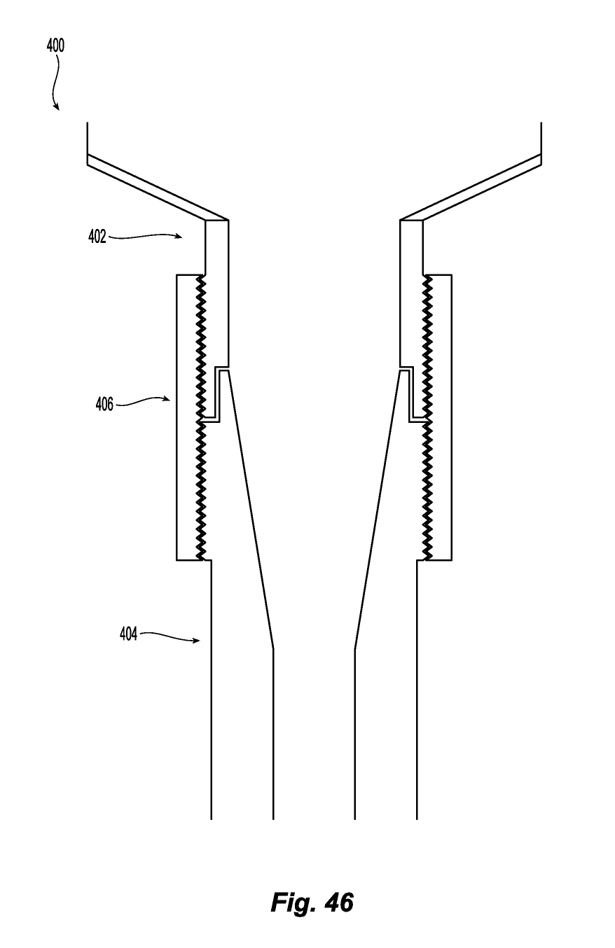

Turning to FIG. 46, a connector 400 between arterial or venous port of the dialysis filter 402 and arterial or venous blood line 404 is shown. The connector has a cylindrical fastener 406 with helical ridge, and with external and internal threads.

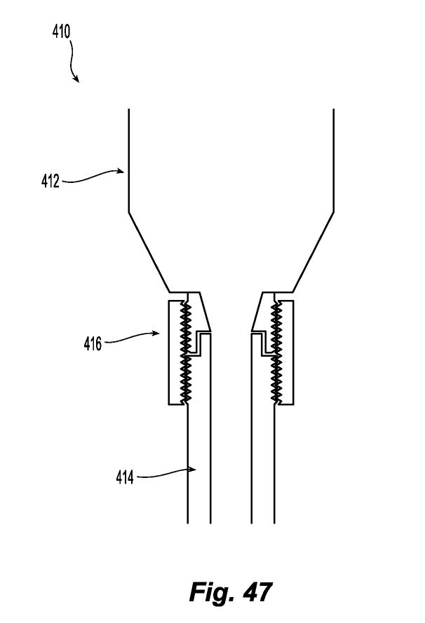

Referring next to FIG. 47, another connector 410 between arterial or venous port of dialysis filter 412 and arterial or venous blood line 414 is shown. This connector is similar to the connector of FIG. 46, having a cylindrical fastener 416 with helical ridge and with external and internal threads, but the internal diameter and shape are different.

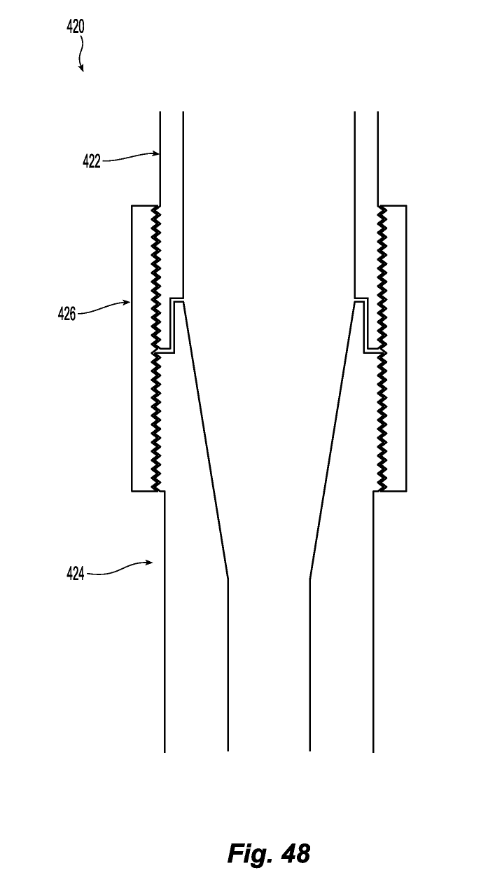

As shown in FIG. 48, a connector 420 is provided between arterial or venous dialysis blood line 422 and arterial or venous fistula needle tubing or arterial or venous port of double lumen dialysis catheter 424 (the connector has the same design as described above). A cylindrical fastener 426 with helical ridge and with external and internal threads is provided.

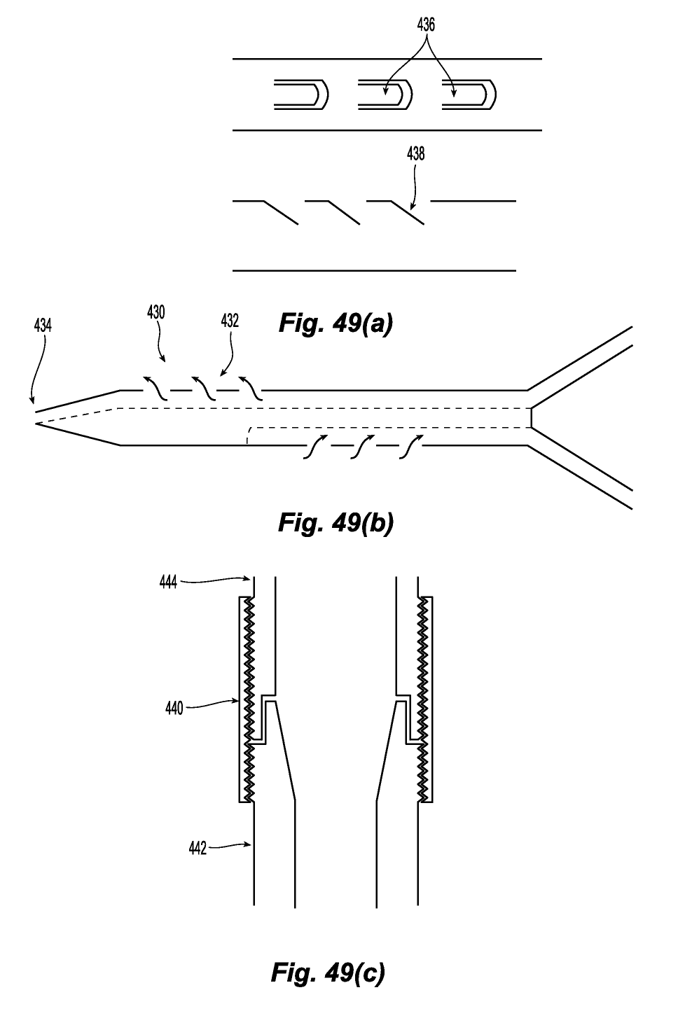

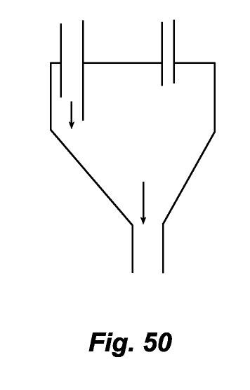

Turning to FIG. 49(a)-(c), a dialysis catheter 430 includes lateral orifices 432 at the venous end. The distal opening 434 may be tapered or not-tapered. Diverters 436, 438 are provided along with a connector 440 between an arterial or venous port 442 of the dialysis catheter and an arterial or venous bloodline 444. Connector 440 includes a cylindrical fastener with helical ridge and with external and internal threads.

Finally, as shown in FIG. 50, another design of an air trap (part of the venous blood line) is shown.

While various descriptions of the present invention are described above, it should be understood that the various features can be used singly or in any combination thereof. Therefore, this invention is not to be limited to only the specifically preferred embodiments depicted herein.

Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A hemodialysis circuit comprising:
a blood flow path between components including a cannula, fistula needle tubing, a blood line, and at least one selected from the group consisting of an artificial kidney and an air trap;
wherein the blood flow path has no sharp transitions between the components.

2. The hemodialysis circuit of claim 1, further comprising at least one connector between two of the components, wherein the connector provides a smooth transition of the internal surfaces of the two components.

3. The hemodialysis circuit of claim 2, wherein at least one free end of the connector is tapered.

4. The hemodialysis circuit of claim 2, wherein the smooth transition is provided by at least one internal arcuate surface of the two components.

5. The hemodialysis circuit of claim 2, wherein the connector is disposed between the fistula needle tubing and blood line.

6. The hemodialysis circuit of claim 2, wherein a free end of the blood line has a larger internal diameter than an external diameter of a free end of the connector, and the free ends of the blood line and connector are directly coupled to one another.

7. The hemodialysis circuit of claim 1, wherein the artificial kidney comprises a port and the blood line is connected directly to the port.

8. The hemodialysis circuit of claim 1, wherein the cannula is received within the fistula needle tubing.

9. The hemodialysis circuit of claim 1, further comprising a blood pump.

10. The hemodialysis circuit of claim 1, wherein the cannula is formed of metal.

11. A system comprising:
at least two components selected from the group consisting of an artificial kidney, an arterial blood line, fistula needle tubing, an arterial fistula needle, a venous blood line, an air trap chamber, a venous fistula needle;
wherein the at least two components are integrally formed.

12. The system of claim 11, wherein the arterial blood line has an internal diameter of at least 3.0 mm.

13. The system of claim 11, wherein the venous blood line has an internal diameter of at least 3.0 mm.

14. The system of claim 11, wherein the arterial blood line has an internal diameter of at least 4.0 mm.

15. The system of claim 11, wherein the venous blood line has an internal diameter of at least 4.0 mm.

16. The system of claim 11, wherein a blood flow path is formed between the at least two components and the blood flow path has a diameter of at least 3.0 mm.

17. The system of claim 11, wherein a blood flow path is formed between the at least two components and the blood flow path has a uniform diameter.

18. The system of claim 11, wherein the ratio of the internal diameters of two of the at least two components is between 0.8 and 1.2.

19. A system comprising:
at least two components selected from the group consisting of an artificial kidney, an arterial blood line, fistula needle tubing, an arterial fistula needle, a venous blood line, an air trap chamber, a venous fistula needle;
wherein the at least two components are permanently coupled to one another.

20. The system of claim 19, wherein the arterial blood line has an internal diameter of at least 3.0 mm.

21. The system of claim 19, wherein the venous blood line has an internal diameter of at least 3.0 mm.

22. The system of claim 19, wherein the arterial blood line has an internal diameter of at least 4.0 mm.

23. The system of claim 19, wherein the venous blood line has an internal diameter of at least 4.0 mm.

24. The system of claim 19, wherein a blood flow path is formed between the at least two components and the blood flow path has a diameter of at least 3.0 mm.

25. The system of claim 19, wherein a blood flow path is formed between the at least two components and the blood flow path has a uniform diameter.

26. The system of claim 19, wherein the ratio of the internal diameters of two of the at least two components is between 0.8 and 1.2.

27. A hemodialysis method comprising:
circulating blood from an arterial fistula needle through an artificial kidney and to a venous fistula needle, wherein the blood flows through a blood line and fistula needle tubing having the same internal diameter of at least 3.0 mm and without any change in diameter at an interface thereof.

28. The hemodialysis method of claim 27, wherein the blood flows through a blood line and fistula needle tubing having the same internal diameter of at least 4.0 mm.

29. The hemodialysis method of claim 27, wherein a connector is provided at the interface and the connector couples the blood line and fistula needle tubing without any change in diameter therebetween.

30. The hemodialysis method of claim 27, further comprising:
pumping blood by compressing the blood line while blood is disposed therein, wherein the blood line has an internal diameter and is compressed to no less than 50% of the original internal diameter.

31. The hemodialysis method of claim 27, further comprising:
pumping blood by compressing the blood line while blood is disposed therein, wherein the blood line has an internal diameter and is compressed to no less than 30% of the original internal diameter.

32. The hemodialysis method of claim 28, wherein the blood line has an internal diameter and is compressed to no less than 60% of the original internal diameter.

33. The hemodialysis method of claim 28, wherein the blood line has an internal diameter and is compressed to no less than 70% of the original internal diameter.
